(19) United States
(12) Patent Application Publication
(10) Pub. No.: US 2025/0243443 A1
(43) Pub. Date: Jul. 31, 2025

(54) BIOMASS PREDICTIONS/ESTIMATION SYSTEM BASED ON PARAMETER SENSING FOR FIXED BED BIOREACTOR AND RELATED METHODS

(71) Applicant: UNIVERCELLS TECHNOLOGIES SA, Nivelles (BE)

(72) Inventors: Bart Heeren, Nivelles (BE); Antoine Hubert, Nivelles (BE); Bastien Mairesse, Nivelles (BE); Clement Dumont, Nivelles (BE); Guillaume Belhaj, Nivelles (BE); Jean-Christophe Drugmand, Nivelles (BE)

(21) Appl. No.: 18/854,869

(22) PCT Filed: Apr. 7, 2023
(86) PCT No.: PCT/EP2023/059300

§ 371 Date: Oct. 7, 2024

Related U.S. Application Data
(60) Provisional application No. 63/412,709, filed on Oct. 3, 2022.
(60) Provisional application No. 63/410,252, filed on Sep. 27, 2022.
(60) Provisional application No. 63/330,967, filed on Apr. 14, 2022.
(60) Provisional application No. 63/328,461, filed on Apr. 7, 2022.

(51) Int. Cl. C12M 1/12; C12M 1/00; C12M 3/00

(52) U.S. Cl. CPC C12M 25/18; C12M 23/06; C12M 23/34; C12M 23/38; C12M 23/48; C12M 25/02; C12M 37/04

(57) ABSTRACT

A bioreactor includes a housing with a number of removable parts for modular construction and easy assembly or disassembly. The removable parts include a fixed bed and a support for the fixed bed. More than one support may be provided. The multiple supports may interlock with each other or a lid of the bioreactor. The support interlocks with the housing to prevent relative rotation. The multiple supports include a support frame for supporting the fixed bed an allowing fluid to flow through the support frame. The fixed bed forms a peripheral chamber between the support and the housing to hold the fixed bed. One or more probes and tubes may be inserted into the bioreactor to test parameters of or to add to or remove liquid from inside.

This application claims the benefit of U.S. Provisional Patent Application Ser. Nos. 63/328,461, filed on Apr. 7, 2022, 63/330,967, filed on Apr. 14, 2022, 63/410,252, filed on Sep. 27, 2022, and 63/412,709 filed on Oct. 3, 2022, the disclosures of which are incorporated herein by reference. This application also relates to U.S. Provisional Patent Application Ser. Nos. 62/758,152, 62/733,375, and 62/608,261, each of which is incorporated herein by reference. The disclosures of U.S. Patent Application Publication No. 2018/0282678, International Patent Application PCT/EP2018/076354, U.S. Provisional Patent Application 62/711,070, and U.S. Provisional Patent Application 62/725,545 are also incorporated herein by reference.

### TECHNICAL FIELD

This document relates generally to the cell culturing arts and, more particularly, to a modular bioreactor, a bioreactor system, and related methods.

### BACKGROUND

Bioreactors are frequently used for culturing cells. Oftentimes, bioreactors include complicated housing arrangements formed of multiple parts requiring excessive welding or gluing to assemble. This can increase the complexity of manufacture, risks for failure, and ultimately the cost to the end user.

A further issue relates to the ability to maximize cell density in the cell growth area of a bioreactor. Many past proposals for fixed bed bioreactors use packed beds. While such packed beds may work well for promoting cell growth and provide certain advantages, the resulting volume of space in the bioreactor required to create such a bed is large. Readily scaling a bioreactor with an unstructured packed bed or fluidized bed while achieving the desired cell growth is also challenging, and there is a current demand for bioreactors that may be utilized in a variety of operating conditions in the field (including, for example, within a sterile hood, where clearance may be limited).

Cell culturing may be expensive, both in terms of time and resources involved in a given process. Failure of such an effort, or sub-optimal conditions for a given process, may result in wasted time and resources, or at the very least a sub-optimal use of available resources (including researcher time, physical components, and various materials involved in a given process). For this reason, small-scale runs may be useful, such as for the purpose of process development, experimental design, evaluating viability of a given process, or for optimizing parameters thereof. If such evaluations are conducted on larger scales, then each run of a given process for experimental purposes utilizes a larger amount of resources.

Additionally, evaluation of process parameters in a single bioreactor system requires either excess time in order for experiments to run sequentially, or additional physical resources in order to control multiple separate single bioreactor systems. For example, evaluating the effect of variations in a given parameter (e.g. temperature, pH, DO, oxygenation/kLa, etc.) or a given set of parameters on a given process could require significant time for sequential runs in order to vary said parameter(s) and/or significant resources and space for simultaneously running the process at various different parameters in order to optimize a process.

Accordingly, a need is identified for an improved bioreactor that would be easy and inexpensive to manufacture, and which could be readily scaled to accommodate a variety of operating conditions. The bioreactor would be capable of being assembled in a highly repeatable manner. A related bioreactor system would also be capable of facilitating scale-up once optimal conditions and/or parameters are determined.

### SUMMARY

In a first embodiment, an apparatus for culturing cells is disclosed, which comprises a bioreactor including a housing having a wall forming an interior compartment, a fixed bed for culturing cells, a fixed bed support for the fixed bed, the fixed bed support adapted to be removably positioned within the interior compartment of the housing, and one or more positioners for uniformly spacing the fixed bed support from the wall of the housing.

In this or other embodiments, the fixed bed support comprises a central portion and a peripheral portion extending radially outward from the central portion and including the one or more positioners, the peripheral portion having an outer diameter corresponding to an inner diameter of the housing. The peripheral portion may comprise one or more projections. The peripheral portion may comprise an outer ring connected to the central portion via the one or more projections.

In this or other embodiments, the peripheral portion comprises an annular disc shaped surface with a plurality of apertures therein. The peripheral portion may comprise a mesh or screen. The peripheral portion may be adapted to support the fixed bed from underneath. The peripheral portion may be adapted to allow fluid flow therethrough.

In another aspect of this or other embodiments, the central portion may include a separator forming a plurality of spaces in an interior of the central portion for accommodating conduits within the housing. The plurality of spaces may be different sizes. At least one of the conduits may be adapted for transmitting fluid flow to or from the interior of the central portion. The separator may be adapted to position the at least one of the conduits adapted for transmitting fluid flow away from an inner wall of the central portion to prevent contact between the conduit and a falling film of liquid running down the inner wall.

In another aspect of this or other embodiments, the central portion of the support forms a container for containing an agitator. The agitator may comprise an impeller adapted to rotate about an agitator support adapted to receive and hold the fixed bed support. The agitator support may comprise a tubular post that connects with a flexible drain tube connected to a lid of the bioreactor.

In another aspect of this or other embodiments, the peripheral portion of the fixed bed support may include one or more projections in the form of a plurality of radially extending arms. The plurality of radially extending arms may connect to a rim having an outer diameter corresponding to an inner diameter of the housing. The plurality of radially extending arms may engage and support the fixed bed.

In a further aspect of this or other embodiments, the housing includes a receiver for receiving at least one of the one or more projections. The central portion and the peripheral portion of the fixed bed support may comprise a single unitary structure.

In another aspect of this or other embodiments, the fixed bed comprises one or more layers of woven or non-woven material wound around the central portion of the fixed bed support.

In another aspect of this or other embodiments, the apparatus may further include a seal for sealing between an inner wall of the housing and the fixed bed support.

In another aspect of this or other embodiments, the fixed bed comprises a plurality of fixed bed portions, the fixed bed support further including a plurality of interlocking support portions for supporting each of the plurality of fixed bed portions. Each interlocking support portion may be adapted for interlocking with an adjacent support portion. The apparatus may further include a first seal for sealing together each adjacent interlocking support portion. The apparatus may further include a second seal for sealing each of the plurality of portions with an inner wall of the housing.

In another aspect of this or other embodiments, the apparatus further includes an upper frame for positioning above the fixed bed. The upper frame may be of a sufficient height to form at least one pocket for allowing fluid to accumulate therein upon exiting an upper end of the fixed bed, and a sensor for sensing a characteristic of the fluid in the at least one pocket. The upper frame may be adapted for engaging a lid of the bioreactor. The upper frame may form a plurality of pockets, the plurality of pockets having a different volume. The upper frame may be adapted for receiving or aligning one or more samplers for sampling the fixed bed.

In a further aspect of this or other embodiments, the apparatus further includes a lid removably connected to the housing. The lid may be adapted to hold the fixed bed vertically in place within the housing. The lid may include a depending portion for engaging the fixed bed or the fixed bed support. The lid may be adapted for threadedly engaging the housing. The apparatus may further include a gasket between the lid and the housing.

In another aspect of this or other embodiments, the apparatus may include a port in the wall of the housing above an upper end of the fixed bed when positioned therein.

In another aspect of this or other embodiments, the housing may comprise a single piece rigid structure forming the interior compartment.

In another embodiment of this disclosure, an apparatus for culturing cells is disclosed, which comprises a housing and lid together defining a container having an interior compartment, and an assembly for positioning within the interior compartment, the assembly including a fixed bed adapted for culturing cells, wherein the assembly is adapted to interlock with the container to retain the position of the fixed bed within the interior compartment.

In one aspect of this or other embodiments, the assembly comprises an upper portion adapted to interlock with the lid. The upper portion may comprise an upper frame for positioning above the fixed bed. The upper frame may be of a sufficient height to form at least one pocket for allowing fluid to accumulate therein upon exiting an upper end of the fixed bed, and a sensor for sensing a characteristic of the fluid in the at least one pocket. The upper frame may include a recess for engaging a projection extending from a lid of the bioreactor. The upper frame may form a plurality of pockets, the plurality of pockets each having a different volume.

In another aspect of this or other embodiments, the fixed bed comprises a plurality of fixed bed portions, each associated with one of a plurality of supports adapted to interlock with an adjacent support. The upper frame may be adapted to interlock with at least one of the plurality of supports. Each of the plurality of supports may comprise a central portion and a peripheral portion adapted to allow fluid to reach the fixed bed, the peripheral portion having an outer diameter corresponding to an inner diameter of the housing.

In another aspect of this or other embodiments, the apparatus may further include at least one O-ring between at least two of the plurality of supports or between the lid and at least one of the plurality of supports. Upon attachment of the lid to the housing, the lid may be adapted to provide downward pressure on the assembly. The downward pressure may be sufficient to maintain the at least one O-ring in place without glue.

In a further aspect of this or other embodiments, the assembly may comprise a lower portion for containing an agitator, the lower portion adapted to interlock with at least one support for supporting the fixed bed.

In a third embodiment of the present disclosure, an apparatus for culturing cells is described, comprising a bioreactor including a housing having a wall forming an interior compartment, a fixed bed for culturing cells, and a support for the fixed bed adapted to be removably positioned within the interior compartment, the support at least partially forming a chamber containing an agitator and including one or more centering projections extending toward the wall of the housing.

In one aspect of this or other embodiments, the projections engage the fixed bed.

In another aspect of this or other embodiments, the housing includes one or more receivers for receiving the one or more projections. An engagement between the one or more projections and the one or more receivers may be adapted to prevent rotation of the support within the housing.

In a fourth embodiment, an apparatus for culturing cells is disclosed that comprises a housing having a wall forming an interior compartment, a fixed bed for culturing cells within the interior compartment, one or more probes for extending into the interior compartment adjacent to or into the fixed bed, and an upper frame overlying the fixed bed for retaining the fixed bed and organizing the one or more probes.

In one aspect of this or other embodiments, the upper frame includes one or more indicia for indicating a location or orientation of the one or more probes.

In another aspect of this or other embodiments, the one or more probes are attached to the upper frame.

In a fifth embodiment, an apparatus for culturing cells is disclosed that comprises a single piece housing, at least one fixed bed for culturing cells, and a plurality of fixed bed supports adapted to interlock for positioning within the single piece housing.

In one aspect of this or other embodiments, each of the plurality of fixed bed supports comprises an annular portion and a support frame extending radially outward from the annular portion. The support frame may comprise an outer diameter that corresponds to an inner diameter of the housing. The support frame may comprises a generally planar extension. The support frame may be adapted to allow fluid to flow therethrough. The support frame may extend from a bottom of the annular portion. The support frame may comprise a plurality of radially extending arms connected to a peripheral ring. The support frame comprises a mesh or a screen. The support frame may be adapted to support the at least one fixed bed from below. The support frame may be adapted to serve as a base for the positioning of the fixed bed on the fixed bed support.

In another aspect of this or other embodiments, each of the fixed bed supports includes a projection or a receiver for interlocking with a corresponding projection or receiver on an adjacent of the fixed bed supports.

In a sixth embodiment, an apparatus for culturing cells is disclose, comprising a bioreactor including a housing having a wall forming an interior compartment, a fixed bed for culturing cells, an annular fixed bed support for supporting the fixed bed, the fixed bed support adapted to be removably positioned within the interior compartment of the housing and defining a peripheral chamber between the annular fixed bed support and the housing, an impeller for circulating a fluid through the fixed bed within the peripheral chamber, and an impeller support for extending at least partially through the impeller and centering the impeller in the housing, wherein the impeller is attached to the impeller support via a snap fit connection.

In a seventh embodiment, a method of manufacturing a fixed bed bioreactor is disclosed, which includes the step of interlocking one or more fixed bed supports within a single piece housing.

In one aspect of this or other embodiments, the interlocking step comprises interconnecting a first fixed bed support to a second fixed bed support.

In another aspect of this or other embodiments, the method further includes the step of positioning an impeller within a portion of the first fixed bed support.

In another aspect of this or other embodiments, the interlocking step comprises interlocking the second fixed bed support with a lid for covering the housing.

In a further aspect of this or other embodiments, the method further includes the step of forming a first seal between the first and second fixed bed supports.

In another aspect of this or other embodiments, the method further includes the step of forming a second seal between the second fixed bed support and the housing.

In another aspect of this or other embodiments, the method further includes the step of wrapping a fixed bed around each of the fixed bed supports.

In an eight embodiment, a bioreactor for culturing cells is disclosed. The bioreactor includes a housing having a wall defining an interior compartment, a plurality of fixed beds for culturing cells, and a plurality of annular fixed bed supports. Each of the plurality of fixed bed supports is adapted to support a respective at least one of the plurality of fixed beds. Each of the plurality of fixed bed supports comprises an annular section and a support frame extending radially out from the annular section. The support frame has an outer diameter corresponding in size to an inner diameter of the wall of the housing, said support frame being adapted to support at least one of the plurality of fixed beds from underneath and to allow fluid to flow through the support frame. The plurality of fixed bed supports are adapted to interlock with one another to form a peripheral chamber between the plurality of annular fixed bed supports and the wall of the housing, as well as a central chamber within the annular sections.

The bioreactor further includes a lid for connecting to the housing and for sealing the plurality of fixed beds and the plurality of fixed bed supports in the interior compartment, a plurality of probes extending into the interior compartment adjacent to or into at least one of the fixed beds, and an upper frame overlying the plurality of fixed bed supports and forming a plurality of pockets for allowing fluid to accumulate therein upon exiting an upper end of the plurality of fixed beds. At least one of the plurality of probes is adapted for sensing a characteristic of the fluid in a respective one of the plurality of pockets.

The bioreactor further includes an impeller for circulating fluid within the bioreactor and a container for containing the impeller. The container comprises a plurality of openings adapted to allow fluid to flow from within the container to the peripheral chamber, and a plurality of positioners in the form of radially extending arms extending therefrom and adapted to position the container within the housing and space the container from the wall thereof. The upper frame is adapted to interlock with at least one of the plurality of annular fixed bed supports and to interlock with the lid for preventing relative rotation therebetween.

In a ninth embodiment, a bioreactor for culturing cells is disclosed. The bioreactor includes a housing having a wall defining an interior compartment, a removable fixed bed for culturing cells, and a removable fixed bed support adapted to support the fixed bed. The fixed bed support is annular in shape and includes a plurality of arms extending radially outward, the radially extending arms defining an outer diameter corresponding in size to an inner diameter of the wall of the housing for positioning and centering the fixed bed support in the housing. The plurality of arms are adapted to support the fixed bed from below. The fixed bed support forms a peripheral chamber between an outer wall of the fixed bed support and the housing, as well as a central chamber within the fixed bed support. The fixed bed is adapted to be positioned within the peripheral chamber. The housing includes one or more receivers in the wall of the housing for receiving at least one of the plurality of arms, the one or more receivers adapted to support the fixed bed support within the interior compartment and to prevent relative rotation of the fixed bed support within the housing.

The bioreactor further includes a lid for connecting to the housing and for sealing the fixed bed and the fixed bed support in the interior compartment, and at least one probe extending into the interior compartment at a location within the peripheral chamber and above the fixed bed.

The bioreactor further includes an impeller adapted to rotate on an impeller support, the impeller for circulating fluid within the bioreactor. The impeller is located in a chamber formed between a lower portion of the fixed bed support and a floor of the housing. The impeller is adapted to circulate fluid from the central chamber of the fixed bed support and outward to the peripheral chamber and up through the fixed bed therein.

The bioreactor further includes a drain tube connected to the impeller support for draining the liquid from the bioreactor.

### BRIEF DESCRIPTION OF THE DRAWING FIGURES

The features of the disclosure are set forth with particularity in the appended claims. A better understanding of the features and advantages of the present disclosure will be obtained by reference to the following detailed description that sets forth illustrative embodiments, in which the inventive principles are utilized, and the accompanying drawings of which:

FIG. 1 is a perspective view of a first embodiment of a bioreactor according to one aspect of the disclosure.

FIG. 1A is a cross-sectional view of the bioreactor of FIG. 1.

FIG. 2 is an exploded view of the bioreactor of FIG. 1.

FIG. 3 illustrates a spiral fixed bed for possible use in connection with a bioreactor.

FIGS. 3A, 3B, and 3C illustrate particular details of the spiral fixed bed.

FIGS. 3D, 3E, 3F, and 3G illustrate alternative arrangements for forming a structured fixed bed.

FIGS. 4 and 4A illustrate a container that houses an agitator within the bioreactor of FIG. 1.

FIGS. 4B and 4C illustrate an impeller assembly for use in a bioreactor according to an aspect of this disclosure.

FIG. 5 is an exploded perspective view of fixed bed and a support for the fixed bed according to an aspect of this disclosure.

FIG. 6 is a perspective view of the support with the fixed bed of FIG. 5.

FIG. 6A is a bottom view of the support of FIG. 5.

FIG. 7 is a side view of the bioreactor of FIG. 1.

FIG. 7A is a cross-sectional view taken along line 7A-7A of FIG. 7.

FIG. 8 is a perspective view of a frame for providing upper support for the fixed bed according to an aspect of this disclosure.

FIG. 9 is a perspective cross-sectional view of the bioreactor of FIG. 1.

FIG. 9A is an enlarged view of a portion of the bioreactor of FIG. 9.

FIG. 10 is a partially cross-sectional view of a further embodiment of a bioreactor according to another aspect of this disclosure.

FIG. 10A is an exploded perspective view of the bioreactor of FIG. 10.

FIG. 11 an exploded perspective view of a further embodiment of a bioreactor according to another aspect of this disclosure.

FIG. 11A is a top view of the bioreactor of FIG. 11.

FIG. 11B is a cross-sectional side-view of the bioreactor taken along line 11B-11B of FIG. 11A.

FIG. 12 is another side view of the bioreactor of FIG. 11.

FIG. 12A is a cross-sectional view taken along line 12A-12A of FIG. 12.

FIG. 13 is a partially cutaway cross-sectional view of a lower portion of the bioreactor of FIG. 11.

FIG. 14 is a cross-sectional view of the bioreactor of FIG. 11.

FIG. 15 is a top view of the bioreactor of FIG. 11.

FIG. 15A is a cross-sectional view taken along line 15A-15A of FIG. 15.

FIG. 15B is an enlarged partial view of FIG. 15A.

FIG. 16 is a flow chart illustrating the steps of assembly of the bioreactor according to this disclosure.

FIG. 17 is a schematic diagram of a bioreactor system.

FIG. 18 is a schematic diagram of sampling locations for cell density of a bioreactor.

FIG. 19 illustrates pH and DO parameters over time in a bioreactor.

FIG. 20 illustrate metabolite levels over time in bioreactors of different sizes.

### DETAILED DESCRIPTION

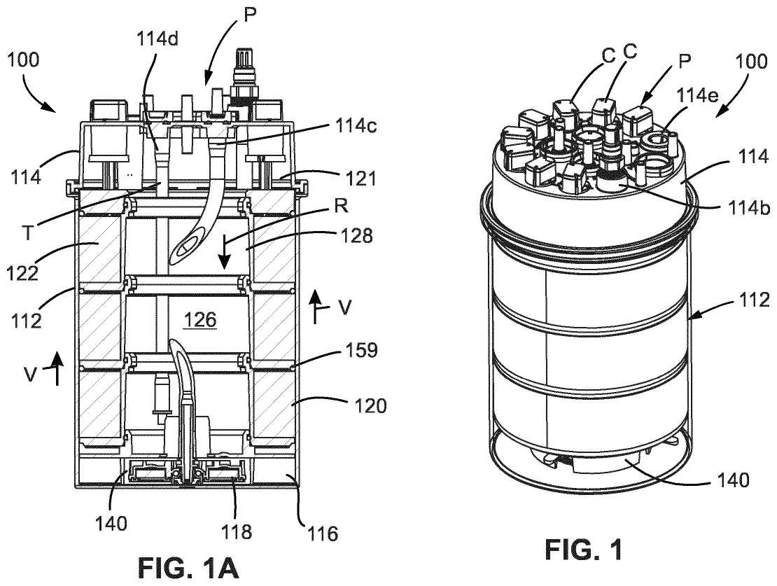

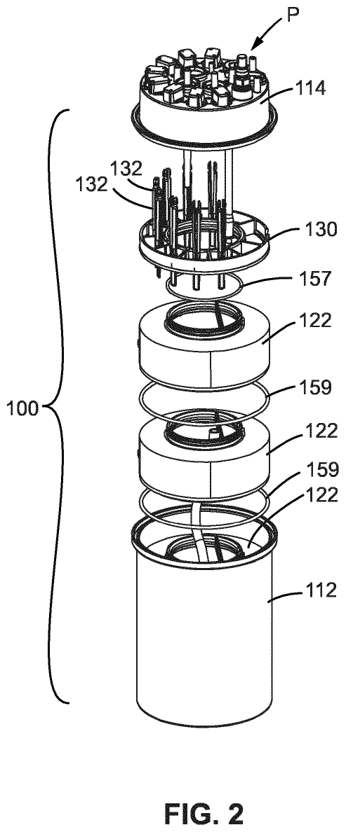

Reference is now made to FIGS. 1, 1A, and 2, which together illustrate one embodiment of a bioreactor 100 for culturing cells according to one aspect of the disclosure. The bioreactor 100 includes an external casing or housing 112, illustrated as transparent in FIG. 1 to allow for perception of inner structures. The housing 112 forms an interior compartment in which cell culturing may be completed using various components or techniques, as outlined further in the following description.

According to one aspect of the disclosure, the housing 112 may in some embodiments form a vessel comprising a single-piece or monolithic structure, such as a pot or bucket having an open top. Providing such a vessel may eliminate the cost and complexity of forming the housing 112 from multiple parts fixed together, such as using welding or adhesives. Furthermore, such a construction avoids the need for associated hermetic seals in the body of the housing 112, thus eliminating the possibility of leakage and/or contamination, and improves bioreactor integrity.

Fabrication of this single piece housing 112 may involve using injection-molding techniques, 3D printing, or other methods such that no seams exist in order to minimize exposure to contamination. In some applications, the housing 112 may be translucent or transparent. In other applications, the housing 112 may be opaque, and may be made of any material, but a preference for plastics exists to allow for a single-use arrangement, if desired.

A cover or lid 114 may overlie the open top of the housing 112 to cover or seal the interior compartment thereof. In one embodiment, the lid 114 is designed to be easily removable, such as by being secured in place by an interlocking engagement with the housing 112 (including possibly a friction-fit or bayonet fitting), but removable fasteners could also be used, such as tabs and/or clips which may interlock with one another, clamps, and/or screws. This facilitates opening the bioreactor 100 and may avoid the need for using samplers (which tend to increase cost and may be challenging to implement in particularly small vessels in view of the size constraints). Together, the housing 112 and the lid 114 may comprise a container for containing the remaining elements of the bioreactor.

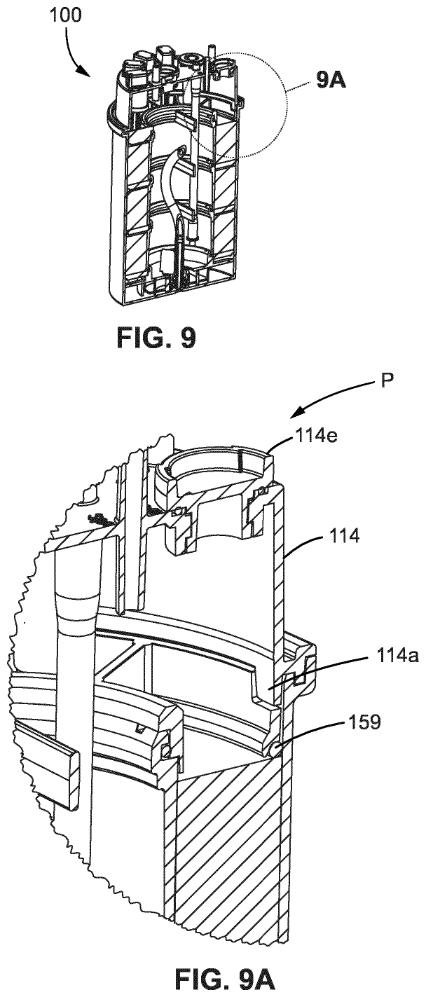

The lid 114 may include various openings or ports P with removable closures or caps C for allowing for the selective introduction or removal of material, fluid, gas, probes, sensors, samplers, or the like, and lends flexibility to the design. In particular, the lid 114 may include holders 114b, such as for receiving suitable sensors (e.g., temperature, capacitance, permittivity, biomass, metabolite such as glucose or lactate, pressure, flow measurement, fluid level, pH or DO probes, or the like). As best shown in FIG. 1A, an internal connector 114c for a conduit or tubing form part of the lid 114. The lid 114 may further include a corresponding connector 114d for a media extraction tube T. As shown in FIGS. 1 and 9A, removable caps 114e with suitable seals, such as O-rings, may permit auxiliary access, if needed. Sampling ports for receiving samplers, such as in the form of probes, may also optionally be provided in the lid 114.

Within the interior compartment formed by the housing 112, several compartments or chambers receive and transmit a flow of fluid, gas, or both, throughout the bioreactor 100. As indicated in FIG. 1A, the chambers may include a first chamber 116 at or near a base of the bioreactor 100. In some embodiments, this first chamber 116 may include an agitator for causing fluid flow within the bioreactor 100. In some embodiments, the agitator may be in the form of a “drop-in” rotatable, non-contact magnetic impeller 118, which thus forms a centrifugal pump in the bioreactor 100. Instead of such an impeller 118, the agitator could also be in the form of a stir bar, an external pump forming part of a fluid circulation system, or any other device for causing fluid circulation within the bioreactor.

The agitation provided results in fluid flowing upwardly (as indicated by arrows V in FIG. 1A) into a second chamber, which may be a peripheral chamber 120 formed in and extending along the outer or peripheral portion of the bioreactor 100. Alternatively, the bioreactor 100 could be adapted to allow fluid to flow in an opposite direct.

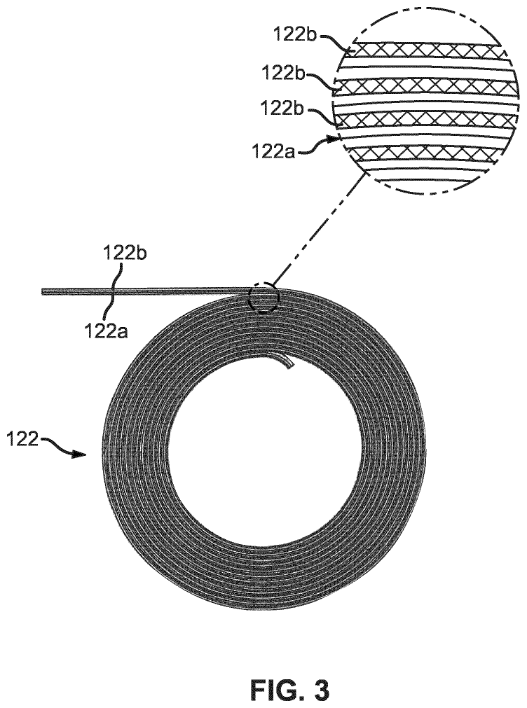

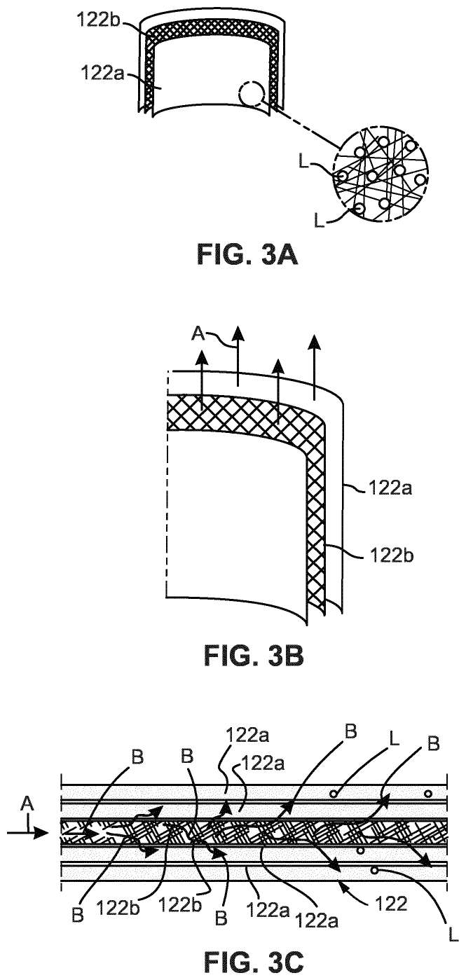

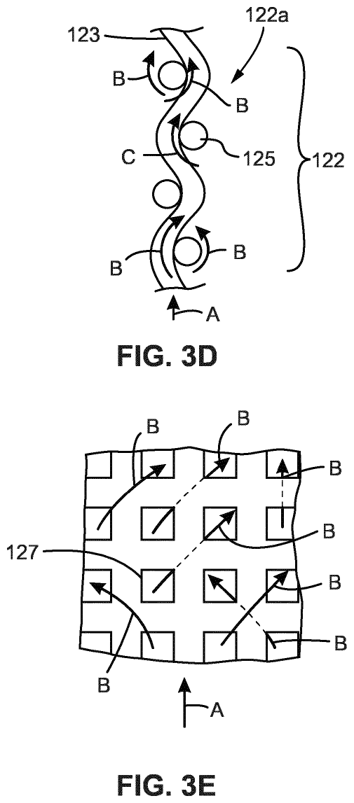

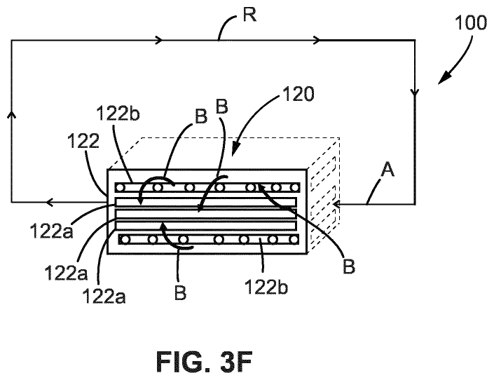

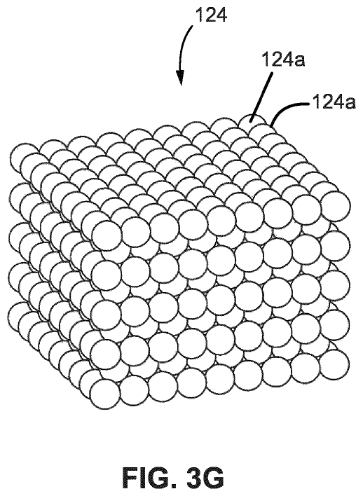

In some embodiments, the bioreactor 100 is adapted to house a cell culture bed 122 in any form including a packed bed, fixed bed, a structured fixed bed, a fluidized bed, etc. For example, FIG. 3 shows a fixed bed 122 in the form of a structured spiral bed, which in use may contain and retain cells being grown. In some embodiments, the spiral bed may be in the form of a cartridge that may be dropped or placed into the peripheral chamber 120, and may be part of a support forming such a cartridge (and adapted for interlocking with additional modular structures, as outlined further in the following description). The bed 122 may be pre-installed in the housing 112 during manufacture at a facility prior to shipping to the end user, or installed at the point of use by the end user.

Fluid exiting the second, peripheral chamber 120 is passed to a headspace formed by an upper chamber 121 on one (upper) side of the bed 122, where the fluid is exposed to a gas (such as oxygen). Fluid may then flow radially inwardly to a third, central chamber 126 to return to the lower portion of bed 122. In some embodiments, this central chamber 126 can be columnar in nature, formed by one or more imperforate conduits or tubes 128 (which may comprise multiple annular portions of fixed bed supports, each including a portion of the fixed bed, as outlined further below), and the flow may be such that a waterfall-like arrangement is created. The central chamber 126 returns the fluid falling or otherwise entering it to the first, base chamber 116 (arrow R showing return path) for recirculation through the bioreactor 100, such that a continuous loop results (“bottom to top” in this version, but such could be reversed or otherwise modified without departing from the disclosure).

As perhaps best understood from the exploded view of FIG. 2, an upper frame 130, such as a spider, may also be provided. The upper frame 130 may be positioned over the upper end portion of the fixed bed 122, such as for example below and adjacent the lid 114. The upper frame 130 may serve to organize structures extending from the lid 114 into or adjacent the fixed bed 122, such as one or more probes 132 that communicate with ports P. In one aspect, the probes or other structures extending in from the lid may contact or be attached to a portion of the upper frame 130. In another aspect, the probes or other structures may be spaced away from the upper frame so as to avoid contact with the upper frame. If present, these probes 132 may serve to sense conditions and/or obtain samples from within the bioreactor 100, and fixed bed 122 in particular, in order to determine the cell growth characteristics without the need to remove the lid 114, if desired.

FIG. 3 shows one embodiment of a matrix material for use as a structured fixed bed 122 in the bioreactor of the present disclosure and, in particular, a spiral bed. In some embodiments, one or more cell immobilization layers 122a may be adjacent to one or more optional spacer layers 122b, which may comprise a woven or non-woven mesh structure. In some embodiments, the layering may optionally be repeated several times to achieve a stacked or layered configuration.

The mesh structure included in spacer layers 122b forms a tortuous path for cells (see cells L in FIG. 3A suspended or entrapped in the material of the immobilization layer 122a), and a cell culture may form part of any invention claimed herein) and fluid to flow through a channel thus created when layered between two immobilization layers 122a. Homogeneity of the cells is maintained within the structured fixed bed as a result of using this type of arrangement. In some embodiments, other spacer structures can be used which form such tortuous paths.

As shown in FIGS. 3, 3A and 3B, the structured fixed bed 122 may be spirally or concentrically rolled along an axis or core (e.g., conduit or tube 128, which may be provided in multiple component parts). In some embodiments, the layers of the structured fixed bed are firmly wound, but could be loosely wound. In some embodiments, the diameter of the open central portion for receiving the tube 128, the length and/or amount of the layers will ultimately define the size of the assembly or matrix. In some embodiments, thickness of each of the layers 122a, 122b may be between 0.1 and 5 mm, 0.1 and 10 mm, or 0.001 and 15 mm.

In some embodiments, other structures can be used which form such tortuous paths. For example, FIG. 3D shows that the one or more cell immobilization layers 122a may be adapted to form a structured fixed bed 122. The one or more layers 122a provide a tortuous channel of flow (arrow B) from a linear or regular inflow (arrow A) without using additional spacer layers (but such may be used, if desired). This may be achieved, for example, by providing a layer of woven fibers or filaments 123, 125 that disrupt the flow.

FIG. 3E shows that such a result may be achieved using a woven or non-woven material as the cell immobilization layer 122a. This may be achieved by forming this layer 122a as a reticulated arrangement (such as by 3-D printing) with openings 127 through which liquid may pass and return again, thus forming the tortuous channels that again promote homogeneity and also serve to further shear or divide any bubbles present in the liquid. This function may again be achieved with or without added spacer layers being present.

The orientation of the structured fixed bed 122 may be other than as shown in a bioreactor 100 as shown in FIGS. 1, 1A, and 2, where the flow is arranged vertically (bottom to top, in the example provided-see arrows V and R). For example, as shown in FIG. 3F, the bioreactor 100 may include the chamber 120 with the structured fixed bed 122 formed of one or more horizontally arranged material layers. The one or more layers may comprise a woven or reticulated material, as per FIGS. 3D and 3E, but as illustrated in FIG. 3F, may comprise one or more cell immobilization layers 122a (three shown, but any number may be present) sandwiched by adjacent spacer layers 122b (vertical spacing exaggerated for purposes of illustration). The flow is thus arranged from side-to-side (left to right or right to left), with the material layer(s) (spacer or otherwise) providing for the channels for creating the tortuous flow (arrows B) from a linear or regular inflow (arrow A). The pumping action may be provided by an agitator or other pump at the entrance end of chamber 120, and a return path provided at the exit end, as schematically illustrated by path R. Additional spacer layers may extend between the cell immobilization layers 122a, if desired.

In another possible embodiment, and with reference to FIG. 3G, the structured fixed bed 122 comprises a three-dimensional (3D) monolithic matrix 124 in the form of a scaffold or lattice formed of multiple interconnected units or objects 124a, which have surfaces for cell adhesion. Preferably, the matrix includes a tortuous path for fluid and cells to flow therethrough when in use. In these or other embodiments, the matrix may be in the form of a 3D array, lattice, scaffolding, or sponge. In all cases, the matrix 124 may be single use in nature to avoid the cost and complexities involved in cleaning according to bioprocessing standards.

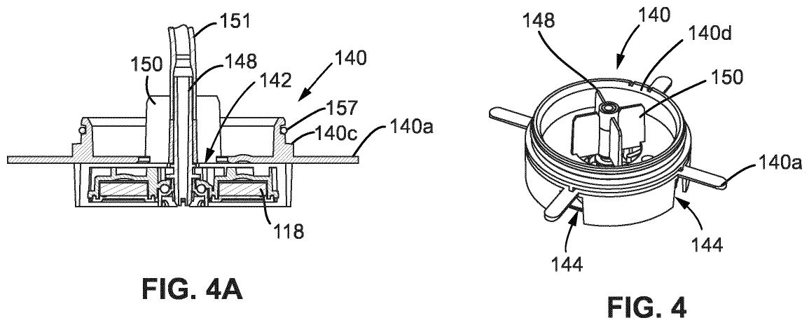

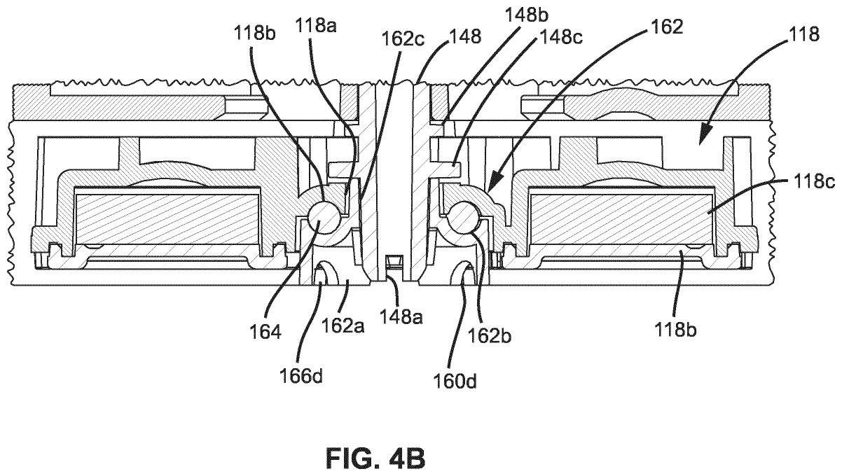

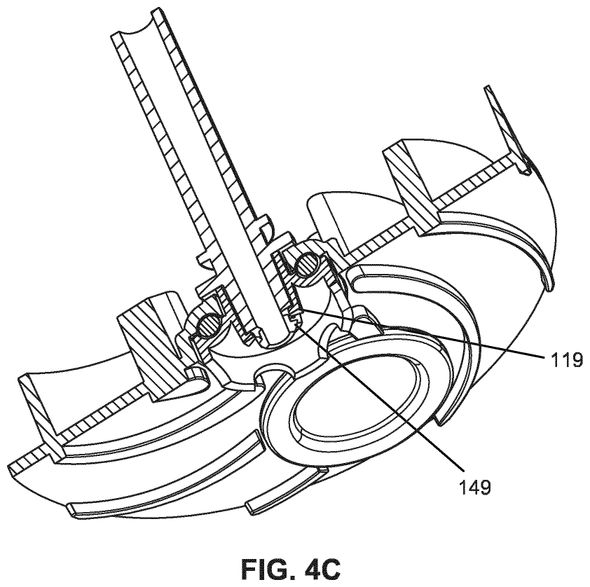

With reference to FIGS. 1, 1A, 4 and 4A, the bioreactor 100 of this first exemplary embodiment may include a support for supporting the fixed bed. In one form, this support may comprise a container 140 for containing the agitator, such as impeller 118, in an interior compartment of the housing 112. The container 140 may be adapted to receive fluid from a central opening 142 and eject the fluid radially outwardly via one or more openings 144 (e.g., four spaced 90 degrees apart), such as a result of the movement (rotation) of the agitator, such as impeller 118.

The container 140 may further include one or more outward projections, which serve as positioners for centering or uniformly spacing the container from an inner wall of the housing 112, but without being attached to it. For example, the container 140 along an upper portion may include one or more radially extending arms 140a. These arms 140a may be adapted for aligning or centering the container within the housing 112 of the bioreactor 110 when rested on a surface thereof, such as the floor. While the arms 140a may be on the container 140, the arms may instead attach to the inner wall of the housing 112 and extend toward the container, but not attach to it, to facilitate easy removal.

FIGS. 4A and 4B illustrates that impeller 118 is adapted to rotate about a support, which may take the form of a tubular post 148. In one aspect, the tubular post 148 may be attached to a bottom of the housing 112. In another aspect, the tubular post 148 may be removable from the housing 112. An upper portion of this post 148 may include one or more vanes 150, and may connect to a conduit 151. The conduit 151 may comprise a flexible tube, and may be used either to supply a gas to the container 140, or else may serve as a drain to withdraw fluid therefrom. In the latter case, the lower portion of the post 148 adjacent to the floor may be crenulated or provided with openings 148a to admit fluid flow. As per FIG. 1A, the conduit 151 may connect at an opposite or upper end to the lid 114, in fluid communication with one of the ports P therein.

As is further shown in FIG. 4B, the lower end of the post 148 may be located in a central opening 118a in the impeller 118. The post 148 may include a first or upper stop, such as a flange 148b, to limit the distance the associated conduit 151 may travel when coupled to the upper end of the post. A second, lower stop, such as flange 148c, may provide an upper limit for movement of the impeller 118. These stops are considered optional, and may take forms other than flanges, as shown.

To facilitate low-friction rotation, a bearing assembly 162 may support the impeller 118. In one example, the bearing assembly 162 may comprise a base 162a including a race 162b for receiving bearings 164, such as in the form of ball bearings, cylindrical bearings, bushing material, or any other bearing element adapted to facilitate relative movement between the impeller. As can be understood, the impeller 118 may have a similar but inverted race 118a forming a compartment when connected to the base 160a, such as by a snap-fit engagement. With reference to FIG. 4C, an example of the snap-fit engagement is illustrated between a projection, illustrated as retractable projection 119 f the impeller 118 and a groove of the post 148.

The impeller 118 may include a compartment with a cover 118b that serves to contain one or more magnets 118c therein. The cover 118b may be removable or permanently fixed in place to seal the compartment with the one or more magnets.

The base 162a may also include an opening 162c for receiving a portion of the post 148. This opening 162c allows the post 148 to reach the floor of the bioreactor 100 within the container 140. The base 160a may also include openings 160d for allowing fluid to enter into it and be drawn into the post 148, such as when negative pressure is applied thereto so as to form a drain, or to release fluid therefrom when supplied under pressure to the post 148 (such as via conduit 151).

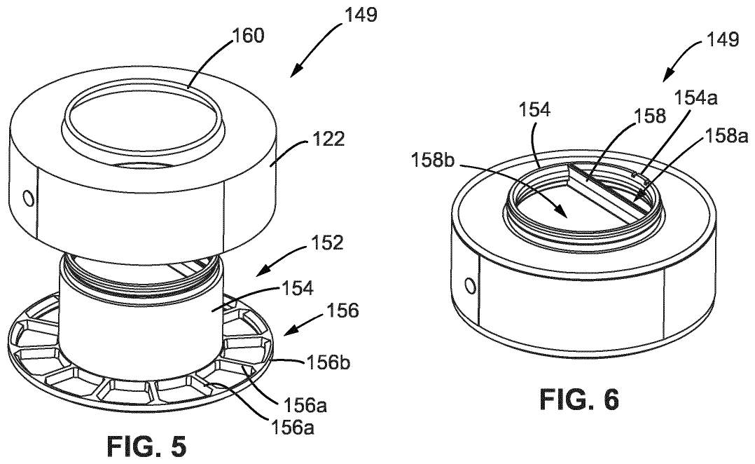

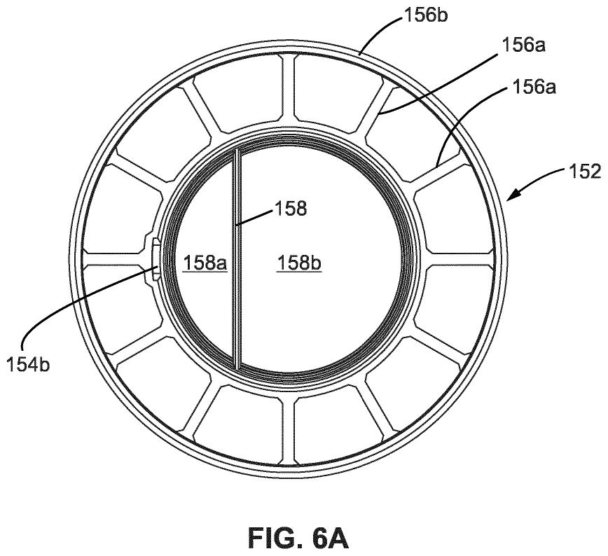

Turning to FIGS. 5 and 6, a fixed bed assembly 149 may be inserted into the interior compartment of the housing 112, which assembly 149 is shown exploded in FIG. 5 and assembled in FIG. 6. The fixed bed assembly 149 may comprise a support 152 for supporting the fixed bed 122 (which may be an entire fixed bed or a portion thereof, such as when a plurality of portions of fixed bed are arranged in a stacked configuration). The support 152 may comprise a one-piece or unitary structure including a central member, shown as an annular portion 154 forming a hollow annular wall corresponding to the imperforate conduit or tube 128 of FIG. 1A, with the material forming the fixed bed 122 spirally wound around it in one example.

The support 152 may further comprise an outer portion, such as a support frame 156, for supporting the fixed bed 122, such as from below in the illustrated arrangement. The support frame 156 may for an extension that extends radially outward from the annular portion 154, but may allow fluid to flow therethrough, such as upward into a lower (inlet) end of the fixed bed 122. The support frame 156 may be located at a bottom of the annular portion 154, such that the support frame 156 provides a level at which a user may wrap or otherwise position the fixed bed 122 around the annular portion 154.

As illustrated, the support frame 156 comprises a plurality of radially extending portions comprising arms 156a connected to a rim or a peripheral ring 156b. In other embodiments, the support frame 156 may comprise an annular shelf or disc, which may include one or more apertures. In some embodiments, the support frame 156 may comprise a mesh or screen. In each of these embodiments, the support frame 156 may perform the dual function of supporting the fixed bed 122 while allowing fluid to flow through the peripheral chamber into the fixed bed.

The support frame 156 has an outer diameter that corresponds to the inner diameter of the housing 112 such that the two structures are directly adjacent to each other and possibly touching, but not connected (so that the fixed bed assembly 149 may be freely inserted into and removed from the interior compartment of the housing 112). Thus, the support frame 156 may function as a positioner or spacer to position or center the fixed bed assembly 149 within the housing 112.

In another aspect, the support frame 156 may serve as a base or reference point for the positioning of the fixed bed 122 on the support 152. For example, if the fixed bed 122 is in the form of a sheet or fabric to be wrapped around the annular portion 154 during use, then the support frame 156 may serve as a level against which at least one edge of the fabric may be laid during the wrapping process. Specifically, if the fixed bed 122 is of a type illustrated in FIG. 3 (or some similar material or combination of materials that may be wound or wrapped around a central core), then the support 152 may be positioned on a surface with the support frame 156 oriented downward, and the fabric or other material(s) forming the fixed bed 122 may be placed such that one edge of the material contacts the support frame 156 and the material forming the fixed bed 122 may be wrapped around the annular portion 154, using the support frame 156 as a guide.

Alternatively, if the fixed bed 122 is pre-fabricated or pre-formed prior to positioning on the support 152 (e.g. an annular disc or series of discs) that is simply inserted over the annular portion 154, then the support frame 156 may provide a stop or floor upon which the pre-fabricated or pre-formed fixed bed 122 may rest.

The annular portion 154 may also include a separator 158, which may divide the interior of it into two (or more) portions 158a, 158b. These portions 158a, 158b may be of the same size and shape, or may be of unequal size and shape. In any case, these portions 158a, 158b may receive, guide and retain in position any tubes or conduits within the interior compartment, such as the conduit 151 for transmitting fluid flow to or from the tubular post 148 associated with the container 140. In one aspect, the separator 158 may be adapted to position a tube or conduit away from an inner wall of the annular support. In this way, a tube adapted for sampling or removing a portion of the liquid within the bioreactor may be held away from the inner wall and prevented from sucking or removing liquid from a falling film of liquid running down the inner wall.

A seal, such as an O-ring 160, may be provided for sealing the annular portion 154 with an adjacent structure, such as another support 152 when a stack is formed. Consequently, a substantially fluid-impervious, or imperforate, conduit or tube 128, may be formed when a plurality of the fixed bed assemblies 149 are stacked within the housing. This may include sealing with the container 140 when present in the bioreactor 100. This may be achieved by a peripheral seal in the form of an O-ring 159 for sealing between the support 152. In particular, the seal or O-ring 159 may seal the peripheral ring 156b and the inner wall of the housing 112 (see FIGS. 1A and 9A which together show such an O-ring 159 seated in a recessed portion of the ring 156b and also in contact with an upper surface of the fixed bed 122 and the inner wall of the housing 112).

When formed in a stack, which is optional, the fixed bed supports 152 may be adapted to interlock, which for purposes of this disclosure means to connect without the need for welding or adhesives, to promote simple assembly and disassembly. Interlocking pieces may connect in a way that limits at least one degree of movement of the interlocked pieces relative to one another. This may include limiting relative rotation between interlocked pieces and/or limiting horizontal or vertical movement between interlocked pieces. For example, this may include one or more projections received within one or more receivers so as to prevent the relative movement.

Specifically, as shown in FIG. 6, the support 152 may include a projection 154a, such as on the upper end of the annular portion 154. Likewise, the support 152 may include a corresponding receiver 154b on an underside of the annular portion 154 or elsewhere thereon. When two adjacent supports 152 are stacked, the projection 154a and receiver 154b thus interlock to align the parts and prevent relative rotation. As can be appreciated, the projection 154a and receiver 154b locations may be reversed with similar effect.

Turning back now to FIG. 4, the container 140 serving as a support for the fixed bed 122 within the housing 112 may include a corresponding projection 140d. Similar to projection 154a, this projection 140d may interlock with the receiver 154b of the fixed bed support 152. Relative alignment and retention of the adjacent fixed bed support 152 may thus be achieved, as well as for any additional fixed bed supports forming a stack in the housing 112, as shown in FIG. 1 (which may comprise any number). A seal, such as an O-ring 157, may seal between the fixed bed support 152 and the container 140, which may also include a seating ledge 140c for receiving and engaging the support (see FIG. 4A).

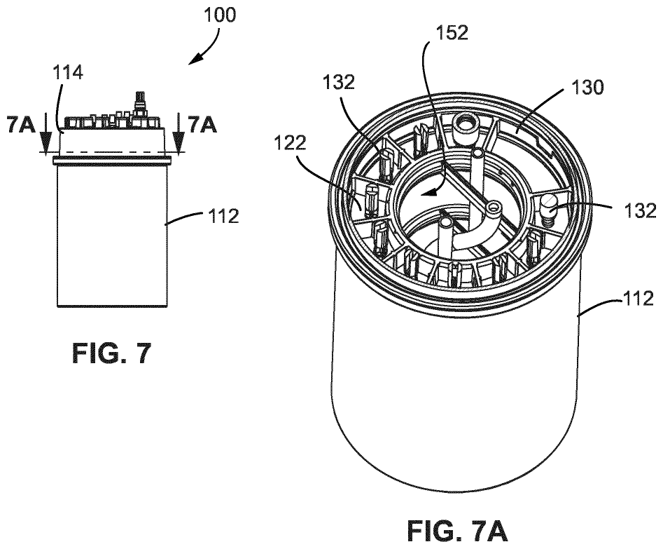

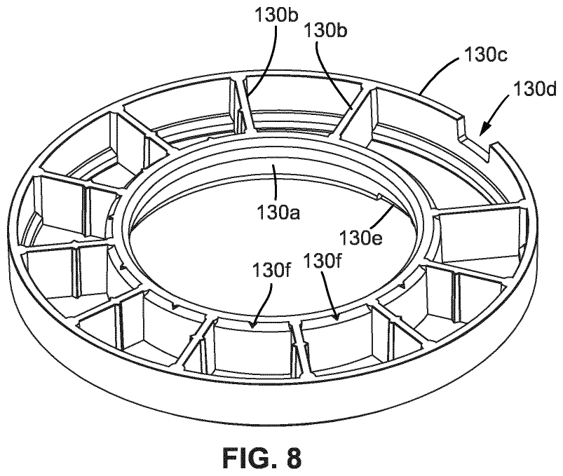

Turning to FIGS. 7, 7A, and 8, further details of the upper frame 130 are illustrated. This upper frame 130 may include an inner member in the form of a central ring 130a connected to an outer member, also in the form of a ring 130c. The connection may comprise a plurality of arms 130b, which may extend radially between the rings 130a, 130c. As can be understood, each adjacent pair of arms 130b forms an opening for allowing fluid to pass, such as from an underlying outlet end of an underlying fixed bed 122 associated with a support 152.

The central ring 130a may have a shape and diameter corresponding to that of the annular portion 154. Likewise, the outer ring 130c may correspond to the inner diameter of the housing 112. An O-ring 157 may seal between the central ring 130a and the upper end of the adjacent fixed bed support 152 (see exploded view of FIG. 2).

As perhaps best shown in FIGS. 8, 9 and 9A, the upper frame 130 may also include features to ensure proper locating or positioning, and which may further serve to provide a retention function. For example, the outer ring 130c may also include a locating feature in the form of a receiver 130d for mating with corresponding locating feature on the lid 114, such as a projection 114a (or receiver), to ensure proper alignment. The upper frame 130 may also include a receiver 130e (or projection) for engaging the projection 140b of a next-adjacent support 152 for the fixed bed. In this manner, the lid 114 secures the upper frame 130, support(s) 152, and container 140 (also serving as a support) together in an interlocking manner, which allows for easy assembly and disassembly of the bioreactor 100.

The upper frame 130 may be dimensioned so as to have a height/depth sufficient to hold a volume of liquid above the fixed bed 122 upon exiting the same and before flowing into the chamber 126 formed by the imperforate conduit or tube 128 established by the annular portion(s) 154 of the supports 152. The fluid well or pocket thus created provides a stable environment for receiving probes/sensors that might otherwise be located in the central chamber 126, and might either be exposed to turbulence or require further insertion to access the liquid therein (which may not completely fill the corresponding chamber). As can be understood from FIG. 7A, these wells or pockets formed by the upper frame 130 may differ in size or volume for different uses, or may have the same size and volume.

As can be further understood from viewing FIGS. 7A and 8 together, the number of openings formed by arms 130b may correspond to the number of structures for passing from the lid 114 to the fixed bed 122, such as no more than one structure (probe) occupies each opening. Thus, as can be understood from FIG. 7A, a plurality of openings along one portion of the upper frame 130 may receive the samplers or probes 132, while others may receive no such structure. To facilitate insertion and alignment during manufacture of the bioreactor 100, the upper frame 130 may include indicia, such as arrows 130f, to identify the proper positioning of the sampler/probe 132 or the like to ensure alignment with a corresponding port P in the lid 114.

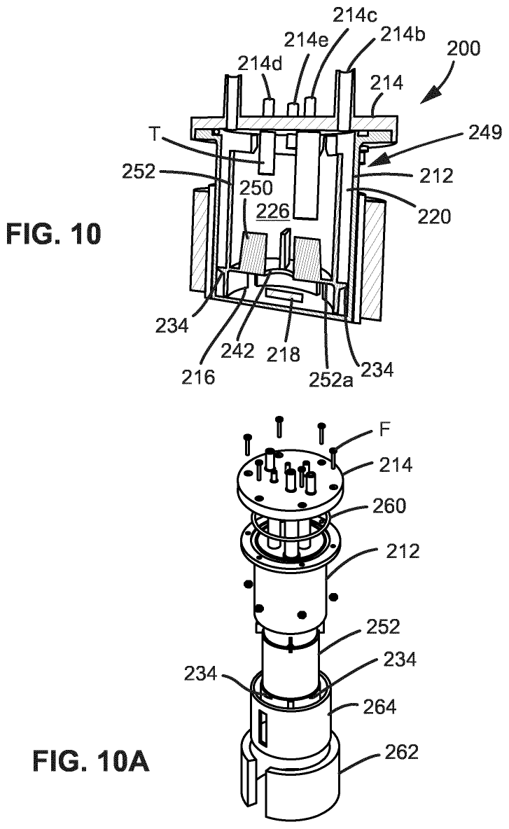

Another example of a bioreactor 200 appears in FIGS. 10 and 10A. In this version, the bioreactor 200 may be similar in structure and arrangement to the larger bioreactor 100 described above, but smaller and simplified in configuration. The structural elements and terms used to describe the bioreactor 200 may be the same as or overlap with the structural elements, terms, and reference numerals of the bioreactor 100 described above, but may differ in some respects, as outlined in the following description (and such differences may be applicable to any disclosed embodiment).

In one example, the bioreactor 200 may include a housing 212 in which a fixed bed assembly 249 including a support 252 for a fixed bed (not shown, but note peripheral chamber 220 as outlined further below in which the fixed bed may reside) may be positioned. The housing 212 may be a unitary piece or a monolithic structure, such as a vessel in the form of a bucket or pot. In such case, the housing 212 may be fabricated using injection molding techniques so that no seams exist, which eliminates leakage and exposure to contamination as compared to multi-part arrangements. In some applications, the housing 212 may be translucent or transparent. In other applications, the housing 212 may be opaque, and formed of any material, but inexpensive plastics are preferred especially if made as a single-use structure.

The support 252 may define a peripheral chamber 220 between the inner surface of the wall of the housing 212 and the outer surface of the wall of the support 252. As illustrated, the housing 212 may be cylindrical, and the support 252 may be annular in shape. Thus, the peripheral chamber 220 may also be annular in shape, and the fixed bed located therein (which may take any form, including those described and shown herein).

The support 252 may include one or more positioners in the form of projections 234. As shown, the projections 234 may comprise radial arms (which may be considered tabs in shortened form) extending toward an inner wall of the housing 212, and possibly engaging but not connecting to it to facilitate separation. Alternatively, the projections 234 may be a part of and extend from the inner wall of the housing 212 toward the support 252, but do not attach to it, still providing the desired spacing between these components.

In one aspect, the bioreactor may include an agitator, such as a magnetic impeller or a stir bar 218. This agitator may be located in a base chamber 216 formed by a lower wall 252a of the support 252 and the floor or base of the housing 212. As illustrated, the lower wall 252a in an interior compartment of the support 252 may include a central opening 242. This central opening 242 may fluidly connect a central chamber 226 of the support 252 with the chamber 216. The chamber 216 may further include one or more sidewalls with apertures or openings that form a fluid pathway with peripheral chamber 220. The support 252 may include the sidewalls of the chamber 216.

Thus, in operation, the agitator, such as stir bar 218, may rotate via an externally applied force via a non-contact (e.g., magnetic) coupling. The agitation serves to draw fluid into the central chamber 226, through the central opening 242 and into the base chamber 216. The fluid may then be forced out through the openings therein, passing over or by the one or more projections 234, and entering the peripheral chamber 220 including the fixed bed. Fluid exiting the peripheral chamber 220 may flow over or through an upper portion of the support 252, back into the central chamber 226, and eventually into the base chamber 216 for recirculation. One or more vanes 250 or baffles may optionally be included within the central chamber 226 to help prevent vortex formation.

The bioreactor 200 may further include a removable screw on lid 214 for sealing the open top of the housing 112. A seal, such as an O-ring 260, may extend between the lid 214 and the housing 212. As an alternative to a screw on lid, the lid 214 may be affixed to the housing 212, such as by fasteners F (e.g., screws, bolts, clamps, clips, tabs), a fitted lip, or any other attachment mechanism for detachably securing the lid in position, and thereby allowing the bioreactor 200 to be reliably sealed during use to guard against contamination, but easily opened.

One advantage of facilitating separation of the lid 214 and the housing 212 is the ability to easily access to the interior of the bioreactor 200, such as prior to, during or after cell culturing. As one specific example, an easy opening lid 214 offers the advantage of a user being able to monitor cell density within the fixed bed (colonization homogenization). In the particular context of a DoE study, the easily opened lid 214 allows, among other things, for simultaneous inoculation of a plurality of bioreactors 200, and then the sequential “sacrifice” of a single bioreactor at a time to evaluate cell density (e.g., one each day) in order to build a kinetic growth pattern of cells. This easy-opening feature facilitates such DoE studies in a way not possible with a more permanent connection between lid and housing, and allows for access to the fixed bed structure to study the cell distribution homogeneity to obtain direct access to cells trapped therein (even in non-sterile conditions).

The lid 214 may include one or more sensor holders 214b. These sensor holders 214b may include one or more disposable sensors for sensing a parameter of the bioreactor 200. In another aspect, the sensor holders 214b may be adapted to receive a reusable sensor for sensing a parameter of the bioreactor 200.

The lid 214 may further include one or more connectors 214c for connecting with a conduit or tubing within the support 252, such as for providing media or any other additive to the bioreactor 200. A further connector 214d may be for connecting to an extraction tube T. One or more removable caps may seal the one or more connectors. The lid 214 may optionally include one or more sampling ports 214e.

With further reference to FIG. 10A, the bioreactor 200 (or bioreactor 100 or any other disclosed version) may include an external temperature regulator. This regulator may take the form of a heating (or cooling) jacket, such as a blanket 262. A thermal conductor, such as a metal (e.g., aluminum) vessel 264, may facilitate heat exchange between the regulator and the bioreactor 200. In one aspect, the blanket 262 and the vessel 264 are not part of the single use bioreactor 200, but rather part of a system.

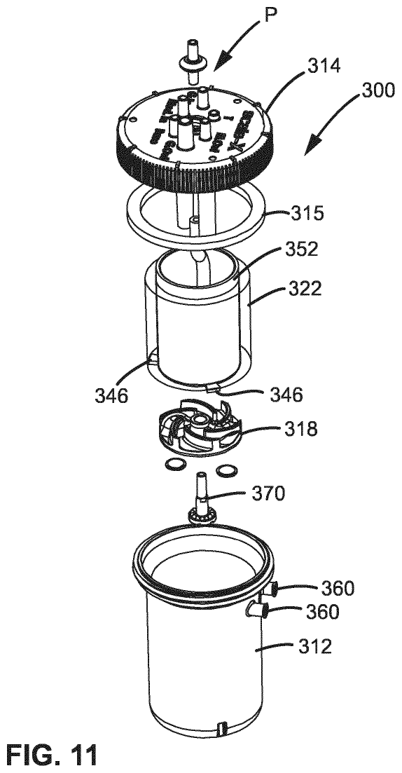

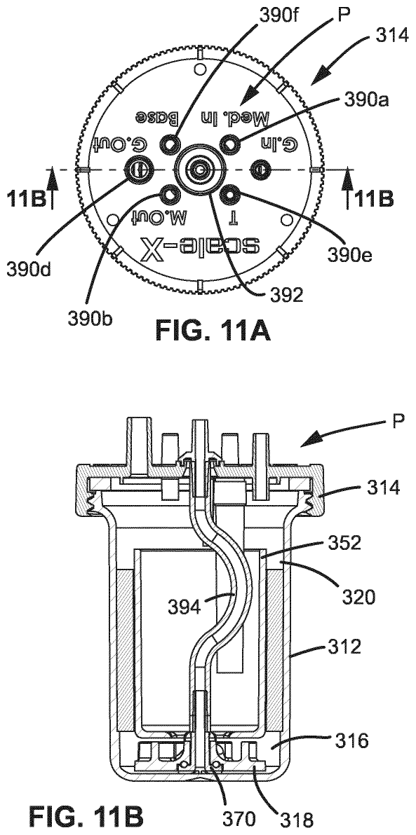

With reference to FIGS. 11, 12, 13, 14, and 15 and the associated portions thereof, as outlined below, another example of a bioreactor 300 is illustrated. As in the example described above, the bioreactor 300 may be similar in structure and arrangement to the bioreactor 100 and/or the bioreactor 200 described above. The structural elements and terms used to describe the bioreactor 300 may be the same as or overlap with the structural elements, terms, and reference numerals of the bioreactor 100 and/or 200 described above, but may differ in some respects, as outlined in the following description (and such differences may be applicable to any disclosed embodiment).

The bioreactor 300 may include a housing 312 including the support 352. In one example, the housing 312 may be a unitary, single-piece or monolithic vessel, such as a canister, bucket or pot. In such case, the housing 312 may be fabricated using injection molding techniques to eliminate seams and the concomitant incidence of leakage or contamination. In some applications, the housing 312 may be translucent or transparent. In other applications, the housing 312 may be opaque, and fabricated of any material, with a preference for plastics if disposability is desired.

Referring to FIGS. 11A and 11B, the support 352 may define a peripheral chamber 320 between the inner surface of the housing 312 and the outer surface of the support 352. As illustrated, the housing 312 may be circular in cross-section, and the support 352 may be annular in shape. Thus, the peripheral chamber 320 may also be annular in shape. The peripheral chamber 320 may be adapted to receive a fixed bed 322. The fixed bed 322 may be any of the types of fixed beds referred to above herein.

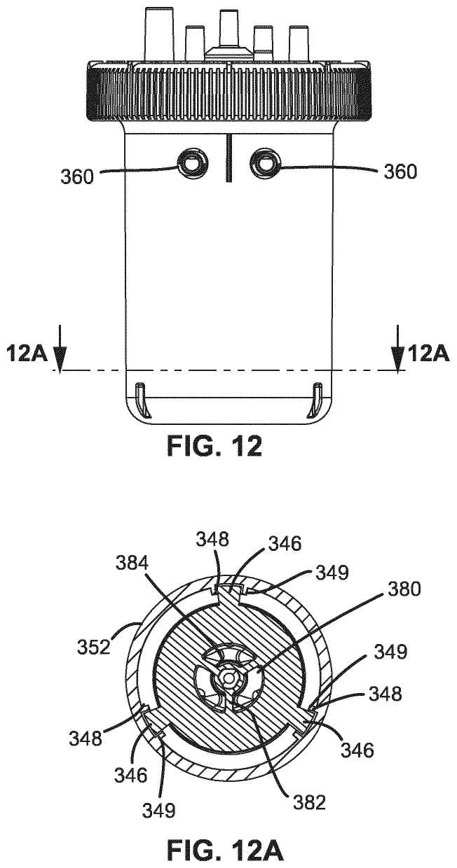

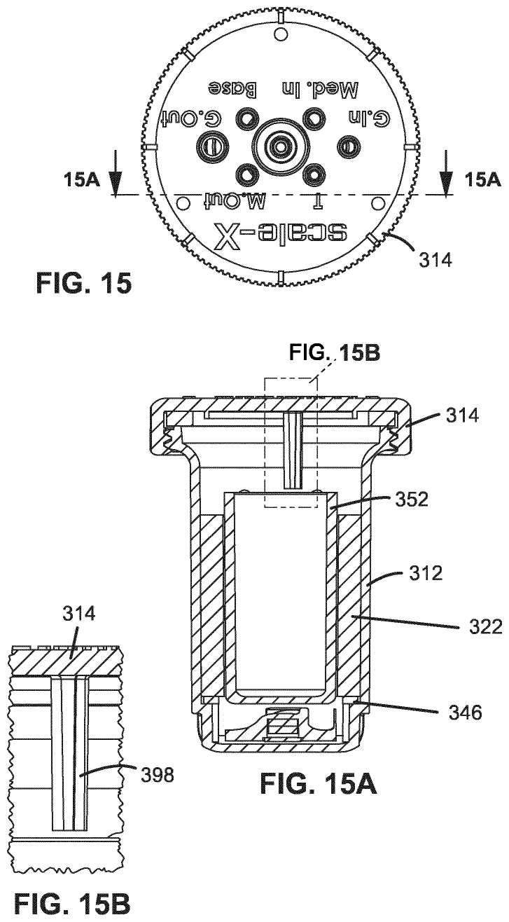

As can be understood from FIG. 11 as well as FIGS. 12 and 12A, one or more positioners, such as projections or arms 346, may position the support 352 within the housing 312. These arms 346 may extend radially outward from a lower portion of the support 352. In one aspect, three such arms 346 are spaced generally equidistant from one another around a circumference of the support 352. These arms 346 may serve to center the support 352 and maintain a fixed distance between the support 352 and the wall of the housing 312, thus at least partially defining a size of the peripheral chamber 320. In another aspect, the arms 346 may support the fixed bed 322 (illustrated as transparent in FIG. 11 for clarity) from below, while still allowing fluid to flow through the peripheral chamber and the fixed bed 322 therein, as perhaps can be best seen in FIGS. 11 and 15A.

The housing 312 may include a receiver 348 adapted to engage at least one of the arms 346. The receiver may be positioned in a lower portion of the housing 312 so as to correspond with a position of the arms 346 of the support 352. The receiver 348 may be in the form of a ledge, shelf, bracket, pocket, slot, recess, or other element adapted to receive and/or prop up and support the arm or arms 346, and therefore the support 352, within the body of the housing 312. The receiver 348 could also take the form of a circumferential lip extending along an inner surface of the housing 312. Alternatively, the positions of the arms 346 and receivers 348 may reverse, such that the arms connect to the housing 312, the receivers 348 connect to the support 352, but remain removably attached to each other.

In one aspect as illustrated in FIG. 12A, the receiver 348 may comprise a plurality of individual receivers, such as a plurality of shelves or ledges, spaced apart from one another and adapted to engage the plurality of arms 346. The receivers 348 may include one or more stops 349 adapted to engage the arms 346 and to restrain the arms 346 from rotating once engaged. For example, the stop 349 may comprise one or more sidewalls associated with the receiver 348. Thus, the receiver 348 may form a pocket or recess adapted to receive the arms 346 and to prevent the support 352 from rotating within the housing 312. In such case, the arms 346 may function to: (1) maintain a space between the support 352 and the wall of the housing 312 (e.g. to maintain a constant dimension of the fluid chamber 320); (2) maintain the support 352 at a desired height within the housing 312; and/or (3) prevent rotational movement of the support 352 within the housing 312.

In another aspect, the positioners or arms 346 may serve as a base or reference point for the positioning of the fixed bed 322 on the support 352. For example, if the fixed bed 322 is in the form of a sheet or fabric to be wrapped around the support 352 during use, then the arms 346 may serve as a level against which at least one edge of the fabric may be laid during the wrapping process. Specifically, if the fixed bed 322 is of a type illustrated in FIG. 3 (or some similar material or combination of materials that may be wound or wrapped around a central core), then the support 352 may be positioned on a surface with the arms 346 oriented downward and the remaining portion of the support 352 oriented upward, and the fabric or other material(s) forming the fixed bed 322 may be placed such that one edge of the material contacts the arms 346 and the material forming the fixed bed 322 may be wrapped around the annular portion of the support 352, using the arms 346 as a guide.

Alternatively, if the fixed bed 322 is pre-fabricated or pre-formed prior to positioning on the support 352 (e.g. an annular disc or series of discs) that is simply inserted over the annular portion of the support 352, then the arms 346 may provide a stop or floor upon which the pre-fabricated or pre-formed fixed bed 322 may rest.

In one aspect, the bioreactor 300 may include an agitator, such as a magnetic impeller 318. The magnetic impeller 318 may be in the form of a “drop-in” rotatable, non-contact magnetic impeller 318, which thus forms a centrifugal pump in the bioreactor. In one aspect, the impeller 318 may include one or more magnet caps, which may be attached (e.g., glued) to ensure that the magnets are sealed onto the impeller. The agitator could also be in the form of an impeller with a mechanical coupling to the base, an external pump forming part of a fluid circulation system, or any other device for causing fluid circulation within the bioreactor. As illustrated, the impeller 318 may comprise a body including curved walls or vanes adapted to draw fluid therein and expel fluid radially outward therefrom.

The magnetic impeller 318 may be positioned in a base chamber 316. In one aspect, this base chamber 316 may be defined at least partially by a lower wall of the support 352 and a lower wall of the housing 312. This base chamber 316 may be in fluid communication with the outer chamber 320. In one aspect, the arms 346 extend between the base chamber 316 and the outer chamber 320.

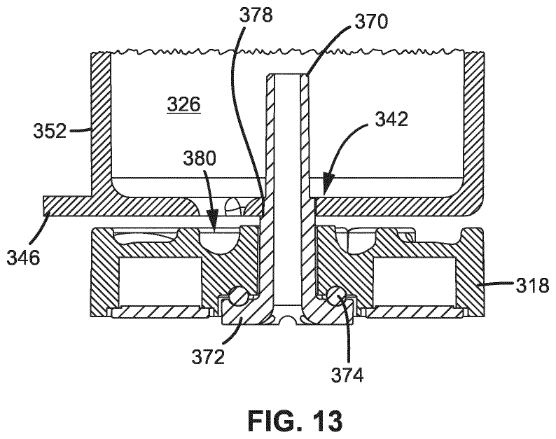

A holder retains the impeller 318 in place which, in the illustrated example, takes the form of a post 370. This post 370 may be adapted to support and maintain a position of the impeller 318 within the base chamber 316. The post 370 may comprise a base 372 adapted to support at least a portion of the impeller 318 from underneath. The post 370 may include a race, which may be adapted to receive a bearing 374 for facilitating rotation of the impeller 318. The bearing 374 may comprise one or more ball bearings, cylindrical bearings, bushing material, or any other bearing element adapted to facilitate relative movement between the impeller 318 and the post 370. In one aspect, the impeller 318 may include a race or a portion of a race for the bearing 374, as shown in FIG. 13 (see also in FIG. 4B for bioreactor 200, but may similarly apply to bioreactor 300). In an aspect of the disclosure, the post 370 may be tubular or hollow, and thus may serve as a conduit for introducing to or withdrawing fluid from the bioreactor 300 via chamber 316, in particular.

As can be seen in FIG. 13, the support 352 may include a central opening 342. This central opening 342 may connect a central chamber 326 of the support 352 with the base chamber 316. The post may be adapted to pass through the central opening 342 of the support 352, thus locating and centering the impeller 318 with respect to the support 352.

The post 370 may include a stop 378 for limiting an insertion distance through the central opening 342. For example, this stop 378 may comprise a shoulder, a lip, detent, outcropping, or other element that may engage with the support 352 and inhibit the post 370 from traveling further through the central opening 342. As illustrated, the stop 378 comprises a first portion of the post 370 with a diameter larger than a diameter of the central opening 342. A second portion of the post 370 may have a diameter smaller than the diameter of the central opening 342, and therefore may pass through the central opening 342 and into the central chamber 326.

The stop 378 may be located at a distance from the base 372 such that a height of the impeller 318 may fit therebetween. In one example, as shown, a distance from the base 372 to the stop 378 is sufficiently greater than a height of the impeller 318 to suspend the impeller 318 therebetween with at least some gap between the impeller and the bottom wall of the support 352. In one embodiment, the post 370 extends below a bottom of the impeller 318 to suspend the impeller 318 above a floor of the housing 312 when the bioreactor 300 is fully assembled.

With reference to FIGS. 12A and 13, the lower wall of the support 352 may include one or more fluid openings 380. Each fluid opening 380 may be adapted to pass fluid from the central chamber 326 to the impeller chamber 316 for distribution to the fluid chamber 320. The fluid openings 380 may be distributed equidistantly about the central opening 342. As shown, the bioreactor 300 includes three fluid openings separated from each other by a separator 382, but any number of fluid openings could be present and any means of providing such openings in the support 352 can be utilized as long as uniform fluid flow is facilitated. The separators 382 may extend from a peripheral portion of the support 352 to a central body 384 of the support 352, which may receive the post 370.

In operation, the impeller 318 may rotate, drawing fluid down through the central chamber 326, through the fluid opening(s) 380 and into the chamber 316. The fluid may then be forced outward and up into and through the peripheral chamber 320 between the support 352 and the housing 312. After fluid rises through the peripheral chamber 320 (which may include the fixed bed 322), the fluid may pass over or through an upper portion of the support 352 and back into the central chamber 326 for recirculation. Although not illustrated in this embodiment of bioreactor, one or more vanes or baffles may prevent the fluid from forming a vortex during recirculation (see, e.g., vanes 260 of bioreactor 200).

The support 352 may be a unitary structure, such as one that may be injection molded. As can be appreciated from the foregoing, this one-piece support 352 may concurrently function as a pump housing or container, a fixed bed support, an anti-vortex structure, form a central chamber for receiving flow (such as a falling film from chamber 220), and prevent movement of all associated components within of the housing 312.

Turning back to FIGS. 11 and 12, and with reference to FIG. 15, the housing 312 may include one or more ports 360 to allow for measuring a characteristic of the bioreactor 300. For example, the ports 360 may be adapted to house or retain a sensor adapted to collect information and/or measure a characteristic of the bioreactor (such as any of pH, dissolved oxygen (DO), temperature, cell density, flow rate, metabolite level, media or media component level, or other parameters, without limitation). In one aspect, the sensor or sensors may be optical sensors.

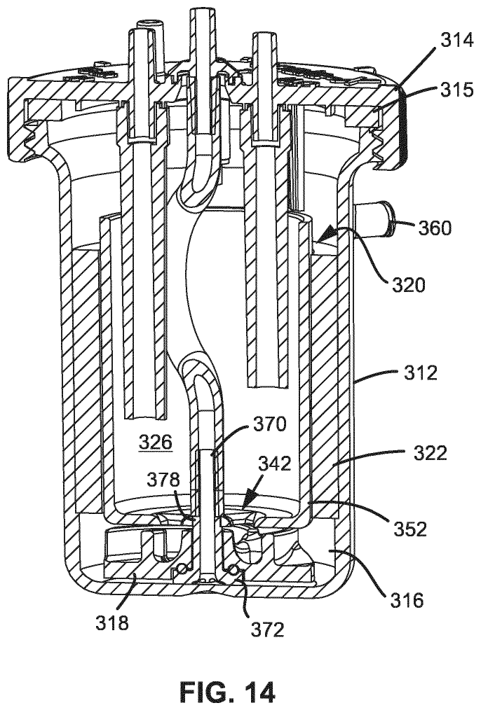

In one embodiment, the ports 360 may be located in communication with the peripheral chamber 320, as illustrated in FIG. 14. More specifically, the ports 360 may be located in an upper portion of the peripheral chamber 320. Even more specifically, the ports 360 may be located in the peripheral chamber 320 at a location above the height of the fixed bed 322. Having the ports 360 at this location places the ports, and therefore the sensors, at a location in the fluid that, under normal flow conditions, has passed through the fixed bed 322.

The ports 360 may comprise a support section, such as a tube or shelf, to support at least a portion of the weight of the sensor. In one aspect, the ports 360 may include a cylindrical hole allowing the sensor direct access to an interior of the bioreactor 300. In another aspect, the ports 360 may be sealed from the interior of the bioreactor, but may allow for non-contact sensors, such as optical sensors, to sense a parameter in the interior of the bioreactor. The ports 360 may include a lock for fixing the sensor in position with respect to the port. The lock may maintain a seal between the inside of the bioreactor 300 and an exterior environment. For example, the ports 360 may comprise a luer lock or other component for maintaining sterility of the bioreactor 300, while allowing the sensor to sense a parameter in the interior of the bioreactor 300.

The bioreactor 300 may further include a lid 314 for sealing the bioreactor 300. The lid 314 may detachably secure to the housing 312, such as by a threaded connection, as illustrated in FIG. 14. Alternatively, the lid 314 may attach to the housing 312 via one or more fasteners, such as screws, bolts, clamps, clips, tabs, a fitted lip, or any other attachment mechanism that allows for the bioreactor 300 to open easily. Together, the housing 312 and the lid 314 may comprise a container for containing the remaining elements of the bioreactor.

One advantage of using easy-open connections between the lid and the housing is the ability to have easy access to the interior of the bioreactor 300, such as during or after an experimental run. For example, in the context of a fixed bed bioreactor, an easy opening lid offers the advantage of potentially facilitating the monitoring of cell density within the fixed bed (colonization homogenization) through direct measurement. In the context of a DoE study, the easily opened lid allows, among other things, for simultaneous inoculation of a plurality of bioreactors 300, and then the sequential “sacrifice” of a single bioreactor at a time (e.g. one each day) in order to build the kinetic growth pattern of cells over time. This easy-open feature facilitates such DoE studies in a way not possible with a more permanent connection between lid and housing.

A seal, such as an O-ring 315, may create a fluid-tight connection between the lid 314 and the housing 312. In some embodiments, the O-ring 315 and/or a portion of the lid 314 or housing 312 adapted to contact the O-ring 315 may be rugose in order to facilitate grip between elements. In some aspects, the O-ring 315 and/or a portion of the lid 314 or housing 312 adapted to contact the O-ring 315 may be smooth in order to facilitate a proper seal between elements.

With reference to FIGS. 11 and 11A, the lid 314 may include one or more connectors or ports P. These ports P may be adapted to connect to one or more conduits, such as for adding or removing media or any other additive or product or waste to or from the bioreactor 300. One or more removable caps may be provided, in some situations including suitable seals, for sealing the port or ports. These ports may include one or more of a first port 390a for adding media, a second port 390b for removing media, a third port 390c for adding a gas, a fourth port 390d for removing a gas, a fifth port 390e for inserting or holding a probe, such as a temperature probe, and a sixth port 390f for adding a pH adjuster, such as a base.

In some embodiments, a port 392 may allow a user to drain the bioreactor 300. The port 392 may connect to a conduit 394, as can be seen in FIGS. 11B and 14. In one embodiment, the conduit 394 may fluidly connect to the post 370. In such an embodiment, the post 370 may comprise a tube or other hollow space therethrough to which the conduit 394 may be connected. The post 370 may include base 372 with openings to allow for draining the bioreactor 300, or such may instead introduce fluid thereto if operated in reverse.

The conduit 394 may serve a further function in maintaining relative position between parts of the bioreactor 300. Specifically, the conduit 394 extends along the post 370 to prevent its withdrawal through the central opening 342 in the support 352. For example, with reference to FIG. 14, the lower end of the conduit 394 may be placed along the post 370, such as being positioned at the point of contact between the support 352 surrounding the central opening 342. In some embodiments, a connector or other support (not shown) may connect the conduit 394 to the post 370, such as a collar. In other embodiments, the size of the conduit 394 is such that an interference or friction fit prevents it from disconnecting. Thus, the conduit 394 may trap or sandwich the bottom wall of the support 352 between the conduit 394 and the stop 378 of the post. This maintains relative position of the conduit 394, the post 370, and the impeller 318, such that these elements do not disconnect once assembled.

In order to facilitate assembly of the bioreactor 300, the conduit 394 may be of a length greater than the height of the bioreactor 300, or greater than a distance between the bottom of the support 352 and the lid 314 (in the assembled condition). For example, the conduit 394 may be between 10-60% longer than the height of the bioreactor 300. In one embodiment, the bioreactor 300 may be 50 mm tall, and the conduit 394 may be of a length between 55-80 mm. In this way, the post 370 may pass through the impeller 318, with the bearing 374 therebetween. The post 370 may extend through the central opening 342 of the support 352, such as until the stop 378 contacts an underside of the support 352. The conduit 394 may connect to the post 370 from the top such that it prevents vertical movement with respect to the support 352.

As can be appreciated, an assembly including the conduit 394, the support 352, the post 370, the bearing 374, and the impeller 318 may be inserted into the housing 312 as a single unit. This insertion can happen simultaneously with or before insertion of the fixed bed 322 into the housing 312. Once inserted into the housing 312, the other end of the conduit 394 may connect to the port 392, and the lid 314 may be attached to the housing 312.

In other aspects, once the conduit 394, the support 352, the post 370, the bearing 374, and the impeller 318 are inserted into the housing 312, any extra length of the conduit 394 may be extended through a hole or aperture in the lid 314 where the port 392 is located. The extra length of conduit 394 may be cut to length, such as within approximately 10 mm above a level of the lid 314. The free end of the conduit 394 may be attached to the port 392, and then the port 392 may be pushed down into the hole or aperture and affixed to the lid 314, such as with glue (e.g. UV glue), welding, or other fastening means.

With reference to FIGS. 15A and 15B, the bioreactor 300 may be adapted to maintain a position of the support 352 within the bioreactor 300. As illustrated, a depending portion of the lid 314, such as a vertical leg 398, may extend downward from the lid 314. The leg 398 may interlock with or lie adjacent to the support 352. The leg 398 may further be of a length corresponding to a distance from the lid 314 to the top of the annular wall of the support 352 and, particularly, when the lid is fully positioned on the housing 312 as a result of the threaded connection used in the illustrated embodiment.

Thus, when the bioreactor 300 is assembled with the lid 314 in place covering the open top of the housing 312, the leg 398 is adjacent to and may even contact the support 352. In either case, this prevents the support from moving vertically any appreciable distance or floating within the bioreactor 300. As illustrated, the leg 398 may include a substantially flat lower edge for engaging the support 352. However, the leg 398 may include one or more extensions, fingers, grips, clips, brackets, or any other structure for engaging the support 352 and inhibiting it from raising vertically within the bioreactor 300. The leg 398 may also be connected to the support 352 instead for engaging the lid 314.

In other embodiments, instead of or in addition to a leg 398, an upper frame such as is illustrated in FIG. 8 and described above may be positioned between the lid 314 and the support 352 in order to maintain a position of the support 352 within the housing.

As described in previous embodiments, the bioreactor 300 may be equipped with a temperature controller, such as a heating (or cooling) blanket and/or a thermal conductor as described herein. Similarly, the bioreactor 300 may be used for similar purposes in the same way as described herein with respect to the bioreactor 200. In another aspect, the bioreactor 300 may be similar in configuration to the aforementioned bioreactor 100, but on a smaller scale. The more similar the configurations of the bioreactor 300 and the larger bioreactor 100, the more likely the optimized or determined parameters in the small-scale bioreactor will result in a similar result of said process in the larger bioreactor 100.

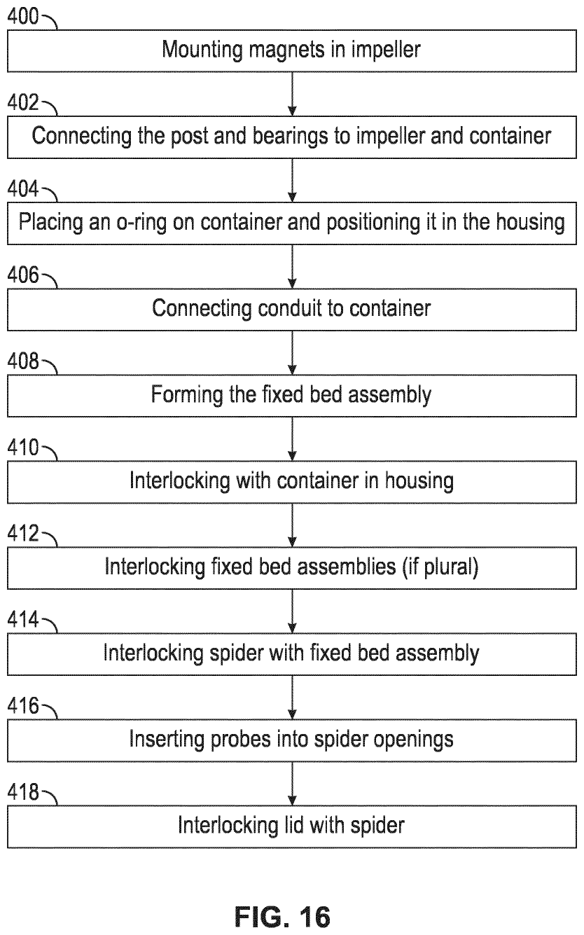

FIG. 16 illustrates in flowchart form exemplary steps for assembling the bioreactor 100 according to the first embodiment of this disclosure. If not previously assembled, the method may include the step 400 of mounting magnets in the body of the impeller 118. Step 402 may include connecting the post 148 and bearings to impeller 318 and container 140. The next step 404 may comprise placing an O-ring on container 140 and positioning it in the housing 112. If present, the conduit 151 connect to the container 140 at step 406.

At step 408, the method may further include forming the fixed bed assembly 149, which in the case of a spiral or wound version may include rolling the fixed bed material(s) onto the support 152 and placing an O-Ring 157 over the container 140. The fixed bed assembly 149 may be placed into the housing 112 to interlock with container 140 at step 410. If multiple stacks are desired, an O-ring 159 may be positioned over the fixed bed 122 of the first fixed bed assembly at step 414, and steps 408, 410, 412 may be repeated as desired.

At step 414, an O-ring 157 is associated with the uppermost fixed bed assembly 149 in the stack to form a seal, and at the upper frame 130 is interlocked with this fixed bed assembly 149. At step 416, samplers or probes may be inserted through the upper frame 130 into the uppermost fixed bed assembly 149 in the desired orientation. If present, the conduit 151 may be connected to the lid 114, which may be interlocked with and sealed to the upper frame 130, as indicating at step 418.

For bioreactors 200 and 300, similar steps may be performed, varying as necessary based on the different arrangement (e.g., since only one fixed bed is provided in the illustrated embodiments for bioreactors 200, 300, the O-rings and repeated assembly steps may be omitted). In any method disclosed, the steps may be implemented or performed by different people or concerns, and may be performed in a different order from that shown. Furthermore, not all steps must be performed and the ordering of steps may vary.

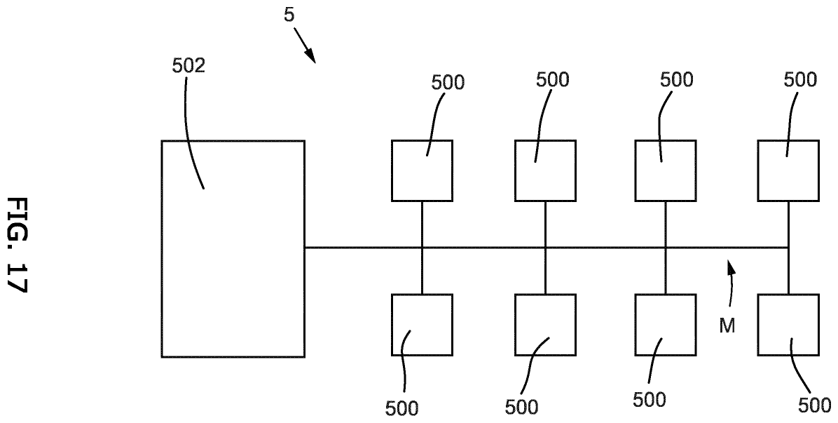

In a further embodiment, and with reference to FIG. 17, it may be advantageous to provide a system S including a plurality of bioreactors 500, such as any bioreactor disclosed herein, simultaneously. In one aspect, a single controller may control aspects of each of the plurality of bioreactors. Such simultaneous control may allow for efficiency of operation or of application of one or more different parameters to a given process in order to optimize conditions for said process. For example, this simultaneous control may allow for control of different cell culture conditions in different bioreactors, including but not limited to pH, DO, temperature, stirring rate, flow rate, etc.

The system S may allow for testing and/or optimization of operational parameters for a given process within the bioreactors 500. Once the desired operational parameters have been determined, it may be desirable to scale the process up from the small-scale bioreactor 200 to a bioreactor capable of running the optimized or desired process at the determined parameters. Thus, in a further aspect of the disclosure, the small-scale bioreactor 200, 300 may be similar in configuration to the larger scale bioreactor 100. The more similar the configurations of the small scale bioreactors 200, 300 and the larger bioreactor 100, the more likely the optimized or determined parameters in the small scale bioreactor will result in a similar result of said process in the larger bioreactor 100.

The bioreactors 500 connect to a single controller 502 forming part of the system S, which is shown with eight bioreactors but any number greater than one may be included. The controller 502 may comprise a computer, a microprocessor, a mobile device, or any other control means adapted to monitor and/or adjust conditions within the bioreactor 500. In one aspect, one or more sensors may be present for monitoring one or more environmental conditions or parameters within a given bioreactor. One or more multiplexors may allow signals, such as sensor and/or control signals, to travel over a single connector such as a wire. This may reduce the amount of elements required for monitoring environmental conditions or parameters (or sending/receiving control signals) within the system.

The system S may be modular, such that one or more bioreactors 500 interconnect to form the system. For example, the system may include a single controller 502 connected to two bioreactors 500. These two bioreactors may function individually or may be a pair of connected bioreactors, and additional single bioreactors may be included. In one aspect, one or more additional pairs of bioreactors may be included. A manifold M may connect to the plurality of bioreactors 200. The illustrated embodiment of FIG. 16 shows four pairs of bioreactors 500, although more or fewer bioreactors or pairs of bioreactors may be included in the system.

The controller 502 may be adapted to monitor and/or control various process parameters in each of the bioreactors 500. For example, the controller 502 may be adapted to control one or more of temperature, pH, DO, stirring speed, or flow rate in a given bioreactor 500. In one aspect, the controller 502 may be adapted to control one or more of the parameters individually in at least one bioreactor, with said parameter being different from the same parameter in another of the bioreactors. In a further aspect, the controller 502 may be adapted to control all of the parameters individually in each of the plurality of bioreactors 500 in the system. Thus, a single controller 502 may be adapted to control a different set of parameters in each of the bioreactors 500. This may allow for a plurality of parallel process conditions to run simultaneously. Such parallel process runs may allow for testing of multiple parameters simultaneously, such as for optimization of conditions for a given process. One advantage of such parallel process runs is that parallel bioreactors may operate using different parameters for DoE (design of experiment) and may guide a user for scaling up of a given culture to a larger bioreactor and/or obtaining data in an efficient manner.

Running a plurality of processes simultaneously may save time, but may not be efficient if conducted on a large scale. Thus, in a further aspect of the disclosure, each bioreactor 500 may comprise a small volume bioreactor, such as a bioreactor with a volume of 300 mL or less. In some examples, the bioreactor may have an operational volume of any of 250 mL, 200 mL, 150 mL, 100 mL, 80 mL, 50 mL, or less. Using a bioreactor 500 of a smaller volume such as this within the system S may allow for efficient use of laboratory space and consumable resources such as media. Additionally, a smaller bioreactor 500 such as this necessarily is smaller in size that a larger reactor, and thus may be more inexpensive to manufacture and therefore more economical to use than a larger suspension reactor. Thus, the use of a plurality of smaller bioreactors 500 may facilitate efficient process development or DoE studies.

In a further aspect, the bioreactor 500 may be a single-use bioreactor, which may include probes or other sensors for measuring parameters thereof. In one aspect, such probes or other sensors may be disposable with the bioreactor 500. In another aspect, the bioreactor 500 may be adapted to receive reusable probes or sensors in a sterile manner. The probes or sensors may include one or more sensors for sensing cell density, optical density, pH, DO, temperature, stirring rate, flow rate (including flow into the bioreactor, flow out of the bioreactor, and flow within the bioreactor, such as during mixing), or other desired parameters associated with cell culturing.

In another aspect, use of a small-scale bioreactor allows for development and testing of a process that may efficiently scale up to a larger reactor. Conversely, a small-scale bioreactor may aid in scaling down a process from a larger bioreactor in order to more efficiently understand or characterize the process. This ability to scale up and/or down may be particularly relevant in the field of viral vectors, as developers and producers work closely together to build large production capacity in a reduced time.

In order to allow for the scale up and/or scale down between reactor sizes, the smaller bioreactor should be representative of the larger bioreactor, in structure and/or function. In the context of a fixed bed reactor, the smaller bioreactor may have a same height and/or compaction of the fixed bed as in the larger bioreactor. In the case of a similar height of fixed bed, a diameter of the fixed bed in the smaller bioreactor may be smaller to reach for a lower surface area. One manner of allowing for scalability in a fixed bed reactor is to maintain structural similarities such as this, along with a similar oxygenation and mixing strategy, which may allow for direct process scale up/down between different sized reactors.

### Example

In a non-limiting example exemplifying scalability between a smaller bioreactor and larger bioreactors, two trial runs conducted in a small bioreactor were used to compare the results to known results of larger bioreactors. The bioreactor conditions utilized are listed in Table 1.

TABLE 1
Bioreactor culture conditions

| | |
|---|---|
| Bioreactors | Scale-X 0.55 m² |
| Cell line | Adherent HEK293 |
| pH | 7.20 |
| DO | >50% |
| Temperature | 37° C. |
| Agitation | 0.5 cm/s |

Feeding strategy

| | |
|---|---|
| DMEM | (4.5 g glucose/L) with 5% FBS Recirculation |
| | loop with 0.17 ml/cm² |
| Growth phase | 5 days |
| Cell seeding | 20,000 cells/cm² |
| Cell cultures | N = 2 |

As a first step, adherent HEK293 cells from a cryopreserved cell bank were thawed. Cells were precultured in T-Flasks, and passaged every 3 to 4 days, (inoculated at 20,000 cells/cm² and harvested in mid-exponential phase) using DMEM media (4.5 g/L glucose/L) enriched with 5% bovine serum and 1% antibiotic-antimycotic.

Prior to being inoculated into the small bioreactor, the cells were further expanded in Cell Factories (Nunc). Cells were then inoculated in the smaller bioreactor (e.g., a scale-X™ bioreactor of the present applicant) with a volume of 0.5 m² at 20,000 cells/cm² and kept for 2-4 h in batch mode to adapt to their new environment before starting the growth phase using a recirculation loop (0.17 ml/cm²) containing the growth media-DMEM (4.5 g/L glucose) enriched with 5% bovine serum. Daily samples of supernatant were taken to evaluate the glucose and lactate profile. Harvest was performed at the end of the run to assess the cell density in the fixed bed.

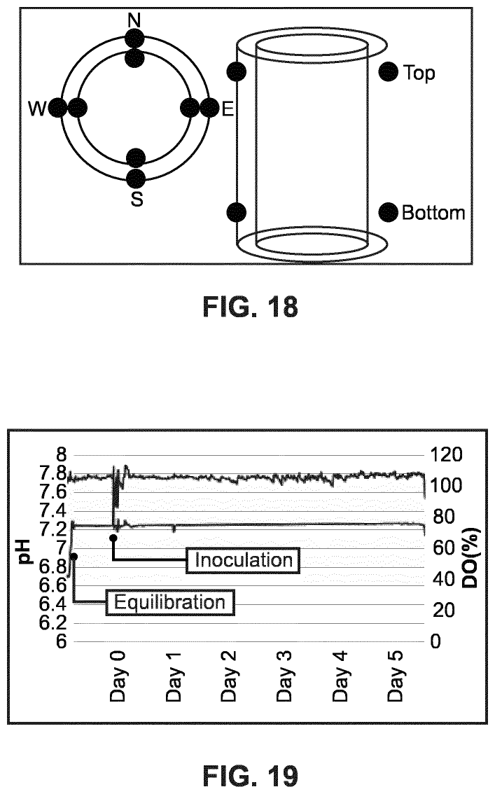

Before retrieving the fixed bed for cell count, the bioreactors were emptied and rinsed with a DPBS solution containing 5 mM of EDTA. The fixed bed was then taken out of the bioreactor. Samples of surface area of 1 cm² each were taken at different positions of the fixed bed (N, S, W, E, on the top and the bottom, on both the internal and external side; see FIG. 18) to estimate cell density and homogeneity.

After each trial run, the density and viability of the suspended cells from the fixed-bed samples were measured by Trypan Blue dye exclusion method using a Thoma hemocytometer. The biomass estimation on the 1 cm² PET samples were performed by acid cell lysis follow by a crystal violet staining and cell nuclei counting. Average cell density reached was 617,032 and 673,932 cells/cm² for run 1 and run 2 respectively, with results summarized in Table 2.

TABLE 2
Bioreactor culture key values; final cell densities
post-dismantling and Population doubling time (PDT)

| Run | Final cell density | Protocol | PDT (h) |
|---|---|---|---|
| 1 | 617 000 cells /cm² | Equilibration D0 Inoc 20k | 29.7 |
| | | cells/cm2 6 days growth | |
| 2 | 673 000 cells /cm² | Equilibration D0 Inoc 20k | 23.6 |
| | | cells/cm2 6 days growth | |

Cell distribution homogeneity was demonstrated through the two runs of the small bioreactor by the cell counts at the different positions on the fixed-bed. Results of these cell counts are presented below in Table 3. At the end of the cultures the maximum difference observed compared to the cell density on the whole fixed bed was low, only slightly exceeding 15% in one location.

TABLE 3
Cell Count at various positions of the fixed bed of the small bioreactor

| | | % vs FB | | cells/cm² % vs | |
|---|---|---|---|---|---|
| Run 1 | cells/cm² | Average | Run 2 | FB Average | |
| FB average | 617032 | N/A | FB average | 673932.125 | N/A |
| Average Layer A | 625593.75 | 1.39% | Average Layer A | 722502.875 | 7.21% |
| Average Layer B | 608470.25 | −1.39% | Average Layer B | 625361.375 | −7.21% |
| North West | 695449.25 | 12.71% | North West | 758851.25 | 12.60% |
| | 499124.25 | −19.11% | | 599853.75 | −10.99% |
| South | 625783.75 | 1.42% | South | 626636.75 | −7.02% |
| East | 647770.75 | 4.98% | East | 710386.75 | 5.41% |
| Top Bottom | 576923.167 | −6.50% | Top Bottom | 714496.1667 | 6.02% |
| | 604862.667 | −1.97% | | 576755.3333 | −14.42% |
| Inside | 698455.5 | 13.20% | Inside | 800939 | 18.85% |
| Outside | 692443 | 12.22% | Outside | 716763.5 | 6.36% |

Furthermore, as shown in FIG. 19, pH and dissolved oxygen (DO) trends maintained good stability during the entire run of the small bioreactors. There were expected peaks or changes in pH and DO during phase chances (e.g. equilibration and inoculation), but overall, the levels remained remarkable steady throughout the run. The pH remained stable between 7.2 and 7.4, respecting perfectly its setpoint. The DO never reached under 100%, indicating that the cell growth oxygenation limit of the bioreactor was never approached.

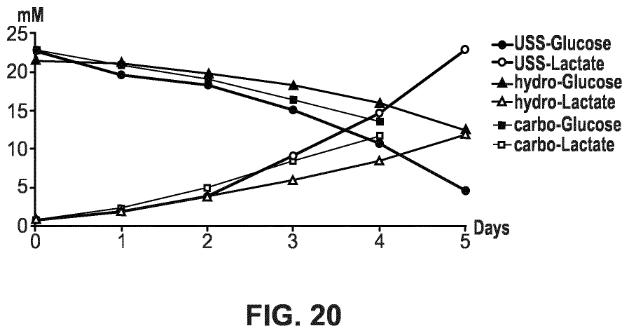

Additionally, various metabolites measured in the small bioreactor during the trial runs were compared to metabolites present during similar runs of larger reactors (e.g., the Scale-X Hydro (2.4 m2), the Carbo (10 m2) products of the present applicant). As shown in FIG. 20, the trends of glucose consumption are comparable between the various reactors during a similarly timed run. In addition, the production of lactate across the various reactors also showed a similar trend.

The small-scale bioreactor 500 has been a successful proof-of-concept showing the easy adaptation of a fixed bed structure to create a small-scale commercial bioreactor of 0.5 m². The testing demonstrated that the same performance as the larger bioreactors in the range may be reliably achieved in a smaller scale version. Cell growth, cell distribution and metabolites behavior data are equivalent to the same process run in the larger scale bioreactors. Demonstrating that the direct scalability can be preserved opens the possibility for new small-scale systems for more efficient and low-cost process development, process optimization and scale-down studies. While the current bioreactor range envisioned spans from growth surface of 2.4 m² to 600 m², this study gives perspective on broadening the range with a smallest size roughly 5× lower than larger versions, reducing development cost, operation and time associated with running a smaller bioreactor.

Summarizing the various aspects to which this disclosure may pertain, the following items are identified, which may be arranged in any combination:

1. An apparatus for culturing cells, comprising:
a bioreactor including a housing having a wall forming an interior compartment;
a fixed bed for culturing cells;
a fixed bed support for the fixed bed, the fixed bed support adapted to be removably positioned within the interior compartment of the housing; and
one or more positioners for uniformly spacing the fixed bed support from the wall of the housing.

2. The apparatus of item 1, wherein the fixed bed support comprises a central portion and a peripheral portion extending radially outward from the central portion and including the one or more positioners, the peripheral portion having an outer diameter corresponding to an inner diameter of the housing.

3. The apparatus of item 2, wherein the peripheral portion comprises one or more projections.

4. The apparatus of item 2 or item 3, wherein the peripheral portion comprises an outer ring connected to the central portion via the one or more projections.

5. The apparatus of any of items 2-4, wherein the peripheral portion comprises an annular disc shaped surface with a plurality of apertures therein.

6. The apparatus of any of items 2-5, wherein the peripheral portion comprises a mesh or screen.

7 The apparatus of any of items 2-6, wherein the peripheral portion is adapted to support the fixed bed from underneath.

8 The apparatus of any of items 2-7, wherein the peripheral portion is adapted to allow fluid flow therethrough.

9. The apparatus of any of items 2-8, wherein the central portion includes a separator forming a plurality of spaces in an interior of the central portion for accommodating conduits within the housing.

10. The apparatus of item 9, wherein the plurality of spaces are different sizes.

11. The apparatus of any of items 9-10, wherein at least one of the conduits is adapted for transmitting fluid flow to or from the interior of the central portion.

12. The apparatus of item 11, wherein the separator is adapted to position the at least one of the conduits adapted for transmitting fluid flow away from an inner wall of the central portion to prevent contact between the conduit and a falling film of liquid running down the inner wall.

13. The apparatus of any of items 2-12, wherein the central portion of the support forms a container for containing an agitator.

14. The apparatus of item 13, wherein the agitator comprises an impeller adapted to rotate about an agitator support adapted to receive and hold the fixed bed support.

15. The apparatus of item 14, wherein the agitator support comprises a tubular post that connects with a flexible drain tube connected to a lid of the bioreactor.

16. The apparatus of item 15, wherein the peripheral portion of the fixed bed support includes one or more projections in the form of a plurality of radially extending arms.

17. The apparatus of item 16, wherein the plurality of radially extending arms connect to a rim having an outer diameter corresponding to an inner diameter of the housing.

18. The apparatus of item 16, wherein the plurality of radially extending arms engage and support the fixed bed.

19. The apparatus of any of items 16-18, wherein the housing includes a receiver for receiving at least one of the one or more projections.

20. The apparatus of any of items 2-19, wherein the central portion and the peripheral portion of the fixed bed support comprise a single unitary structure.

21. The apparatus of any of items 1-20, wherein the fixed bed comprises one or more layers of woven or non-woven material wound around the central portion of the fixed bed support.

22. The apparatus of any of items 1-21, further including a seal for sealing between an inner wall of the housing and the fixed bed support.

23. The apparatus of any of items 1-22, wherein the fixed bed comprises a plurality of fixed bed portions, the fixed bed support further including a plurality of interlocking support portions for supporting each of the plurality of fixed bed portions.

24. The apparatus of item 23, wherein each interlocking support portion is adapted for interlocking with an adjacent support portion.

25. The apparatus of item 23 or item 24, further including a first seal for sealing together each adjacent interlocking support portion.

26. The apparatus of item 25, further including a second seal for sealing each of the plurality of portions with an inner wall of the housing.

27. The apparatus of any of items 1-26, further including an upper frame for positioning above the fixed bed.

28. The apparatus of item 27, wherein the upper frame is of a sufficient height to form at least one pocket for allowing fluid to accumulate therein upon exiting an upper end of the fixed bed, and a sensor for sensing a characteristic of the fluid in the at least one pocket.

29. The apparatus of item 27 or item 28, wherein the upper frame is adapted for engaging a lid of the bioreactor.

30. The apparatus of any of items 27-29, wherein the upper frame forms a plurality of pockets, the plurality of pockets having a different volume.

31. The apparatus of any of items 27-30, wherein the upper frame is adapted for receiving or aligning one or more samplers for sampling the fixed bed.

32. The apparatus of any of items 1-31, further including a lid removably connected to the housing.

33. The apparatus of item 32, wherein the lid is adapted to hold the fixed bed vertically in place within the housing.

34. The apparatus of item 32 or item 33, wherein the lid includes a depending portion for engaging the fixed bed or the fixed bed support.

35. The apparatus of any of items 32-34, wherein the lid is adapted for threadedly engaging the housing.

36. The apparatus of any of items 32-35, further including a gasket between the lid and the housing.

37. The apparatus of any of items 1-36, further including a port in the wall of the housing above an upper end of the fixed bed when positioned therein.

38. The apparatus of any of items 1-37, wherein the housing comprises a single piece rigid structure forming the interior compartment.

39. An apparatus for culturing cells, comprising:
a housing and lid together defining a container having an interior compartment; and
an assembly for positioning within the interior compartment, the assembly including a fixed bed adapted for culturing cells, wherein the assembly is adapted to interlock with the container to retain the position of the fixed bed within the interior compartment.

40. The apparatus of item 39, wherein the assembly comprises an upper portion adapted to interlock with the lid.

41. The apparatus of item 40, wherein the upper portion comprises an upper frame for positioning above the fixed bed.

42. The apparatus of item 41, wherein the upper frame is of a sufficient height to form at least one pocket for allowing fluid to accumulate therein upon exiting an upper end of the fixed bed, and a sensor for sensing a characteristic of the fluid in the at least one pocket.

43. The apparatus of item 41 or item 42, wherein the upper frame includes a recess for engaging a projection extending from a lid of the bioreactor.

44. The apparatus of any of items 41-43, wherein the upper frame forms a plurality of pockets, the plurality of pockets each having a different volume.

45. The apparatus of any of items 41-44, wherein the fixed bed comprises a plurality of fixed bed portions, each associated with one of a plurality of supports adapted to interlock with an adjacent support.

46. The apparatus of any of items 41-45, wherein the upper frame is adapted to interlock with at least one of the plurality of supports.

47. The apparatus of any of items 45-46, wherein each of the plurality of supports comprises a central portion and a peripheral portion adapted to allow fluid to reach the fixed bed, the peripheral portion having an outer diameter corresponding to an inner diameter of the housing.

48. The apparatus of any of items 45-47, further including at least one O-ring between at least two of the plurality of supports or between the lid and at least one of the plurality of supports.

49. The apparatus of any of items 45-48, wherein upon attachment of the lid to the housing, the lid is adapted to provide downward pressure on the assembly

50. The apparatus of item 49, wherein the downward pressure is sufficient to maintain the at least one O-ring in place without glue.

51. The apparatus of any of items 39-50, wherein the assembly comprises a lower portion for containing an agitator, the lower portion adapted to interlock with at least one support for supporting the fixed bed.

52. An apparatus for culturing cells, comprising:
a bioreactor including a housing having a wall forming an interior compartment;
a fixed bed for culturing cells; and
a support for the fixed bed adapted to be removably positioned within the interior compartment, the support at least partially forming a chamber containing an agitator and including one or more centering projections extending toward the wall of the housing.

53. The apparatus of item 52, wherein the projections engage the fixed bed.

54. The apparatus of item 52 or item 53, wherein the housing includes one or more receivers for receiving the one or more projections.

55. The apparatus of any of items 52-54, wherein an engagement between the one or more projections and the one or more receivers is adapted to prevent rotation of the support within the housing.

56. An apparatus for culturing cells, comprising:
a housing having a wall forming an interior compartment;
a fixed bed for culturing cells within the interior compartment;
one or more probes for extending into the interior compartment adjacent to or into the fixed bed; and
an upper frame overlying the fixed bed for retaining the fixed bed and organizing the one or more probes.

57. The apparatus of item 56, wherein the upper frame includes one or more indicia for indicating a location or orientation of the one or more probes.

58. The apparatus of item 56 or 57, wherein the one or more probes are attached to the upper frame.

59. An apparatus for culturing cells, comprising:
a single piece housing;
at least one fixed bed for culturing cells; and
a plurality of fixed bed supports adapted to interlock for positioning within the single piece housing.

60. The apparatus of item 59, wherein each of the plurality of fixed bed supports comprises an annular portion and a support frame extending radially outward from the annular portion.

61. The apparatus of item 60, wherein support frame comprises an outer diameter that corresponds to an inner diameter of the housing.

62. The apparatus of item 60 or item 61, wherein the support frame comprises a generally planar extension.

63. The apparatus of any of items 60-62, wherein the support frame is adapted to allow fluid to flow therethrough.

64. The apparatus of any of items 60-63, wherein the support frame extends from a bottom of the annular portion.

65. The apparatus of any of items 60-64, wherein the support frame comprises a plurality of radially extending arms connected to a peripheral ring.

66. The apparatus of any of items 60-65, wherein the support frame comprises a mesh or a screen.

67. The apparatus of any of items 60-66, wherein the support frame is adapted to support the at least one fixed bed from below.

68. The apparatus of any of items 60-67, wherein the support frame is adapted to serve as a base for the positioning of the fixed bed on the fixed bed support.

69. The apparatus of any of items 59-68, wherein each of the fixed bed supports includes a projection or a receiver for interlocking with a corresponding projection or receiver on an adjacent of the fixed bed supports.

70. An apparatus for culturing cells, comprising:
a bioreactor including a housing having a wall forming an interior compartment;
a fixed bed for culturing cells;
an annular fixed bed support for supporting the fixed bed, the fixed bed support adapted to be removably positioned within the interior compartment of the housing and defining a peripheral chamber between the annular fixed bed support and the housing;
an impeller for circulating a fluid through the fixed bed within the peripheral chamber; and
an impeller support for extending at least partially through the impeller and centering the impeller in the housing;
wherein the impeller is attached to the impeller support via a snap fit connection.

71. A method of manufacturing a fixed bed bioreactor, comprising:

interlocking one or more fixed bed supports within a single piece housing.

72. The method of item 71, wherein the interlocking step comprises interconnecting a first fixed bed support to a second fixed bed support.

73. The method of any of items 71-72, further including the step of positioning an impeller within a portion of the first fixed bed support.

74. The method of any of items 71-73, wherein the interlocking step comprises interlocking the second fixed bed support with a lid for covering the housing.

75. The method of any of items 71-74, further including the step of forming a first seal between the first and second fixed bed supports.

76. The method of item 75, further including the step of forming a second seal between the second fixed bed support and the housing.

77. The method of any of items 71-76, further including the step of wrapping a fixed bed around each of the fixed bed supports.

78. A bioreactor for culturing cells comprising:
a housing having a wall defining an interior compartment;
a plurality of fixed beds for culturing cells;
a plurality of annular fixed bed supports, each of the plurality of fixed bed supports adapted to support a respective at least one of the plurality of fixed beds, wherein each of the plurality of fixed bed supports comprises
   an annular section; and
   a support frame extending radially out from the annular section, the support frame having an outer diameter corresponding in size to an inner diameter of the wall of the housing, said support frame adapted to support at least one of the plurality of fixed beds from underneath and to allow fluid to flow through the support frame;
   wherein the plurality of fixed bed supports are adapted to interlock with one another to form a peripheral chamber between the plurality of annular fixed bed supports and the wall of the housing, as well as a central chamber within the annular sections;
a lid for connecting to the housing and for sealing the plurality of fixed beds and the plurality of fixed bed supports in the interior compartment;
a plurality of probes extending into the interior compartment adjacent to or into at least one of the fixed beds;
an upper frame overlying the plurality of fixed bed supports and forming a plurality of pockets for allowing fluid to accumulate therein upon exiting an upper end of the plurality of fixed beds, wherein at least one of the plurality of probes is adapted for sensing a characteristic of the fluid in a respective one of the plurality of pockets;
an impeller for circulating fluid within the bioreactor; and
a container for containing the impeller, the container comprising
   a plurality of openings adapted to allow fluid to flow from within the container to the peripheral chamber; and
   a plurality of positioners in the form of radially extending arms extending therefrom and adapted to position the container within the housing and space the container from the wall thereof;
wherein the upper frame is adapted to interlock with at least one of the plurality of annular fixed bed supports and to interlock with the lid for preventing relative rotation therebetween.

79. A bioreactor for culturing cells comprising:
a housing having a wall defining an interior compartment;
a removable fixed bed for culturing cells;
a removable fixed bed support adapted to support the fixed bed, wherein the fixed bed support is annular in shape and includes a plurality of arms extending radially outward, the radially extending arms defining an outer diameter corresponding in size to an inner diameter of the wall of the housing for positioning and centering the fixed bed support in the housing, wherein the plurality of arms are adapted to support the fixed bed from below;
   wherein the fixed bed support forms a peripheral chamber between an outer wall of the fixed bed support and the housing, as well as a central chamber within the fixed bed support;
   wherein the fixed bed is adapted to be positioned within the peripheral chamber; and
   wherein the housing includes one or more receivers in the wall of the housing for receiving at least one of the plurality of arms, the one or more receivers adapted to support the fixed bed support within the interior compartment and to prevent relative rotation of the fixed bed support within the housing;
a lid for connecting to the housing and for sealing the fixed bed and the fixed bed support in the interior compartment;
at least one probe extending into the interior compartment at a location within the peripheral chamber and above the fixed bed;
an impeller adapted to rotate on an impeller support, the impeller for circulating fluid within the bioreactor, the impeller located in a chamber formed between a lower portion of the fixed bed support and a floor of the housing, wherein the impeller is adapted to circulate fluid from the central chamber of the fixed bed support and outward to the peripheral chamber and up through the fixed bed therein; and
a drain tube connected to the impeller support for draining the liquid from the bioreactor.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed embodiments. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

While various embodiments have been shown and described herein, it will be obvious to those skilled in the art that such embodiments are provided by way of example only. Numerous variations, changes, and substitutions will now occur to those skilled in the art without departing from the scope of the disclosure. For example, while the bioreactor is shown in a vertical orientation, it could be used in any orientation. In any of the foregoing embodiments, any or all of the components of the bioreactor 100, 200, 300 may be provided as disposable, or “single use” components. This allows for inexpensive manufacture and use, without the need for cleaning and re-sterilization. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1. An apparatus for culturing cells, comprising:
a bioreactor including a housing having a wall forming an interior compartment;
a fixed bed for culturing cells;
a fixed bed support for the fixed bed, the fixed bed support adapted to be removably positioned within the interior compartment of the housing; and
one or more positioners for uniformly spacing the fixed bed support from the wall of the housing;
wherein the fixed bed support comprises a central portion and a peripheral portion extending radially outward from the central portion and including the one or more positioners, the peripheral portion having an outer diameter corresponding to an inner diameter of the housing.

2. (canceled)

3. The apparatus of claim 1, wherein the peripheral portion comprises one or more projections, and wherein the peripheral portion comprises an outer ring connected to the central portion via the one or more projections.

4. (canceled)

5. The apparatus of claim 1, wherein the peripheral portion comprises an annular disc shaped surface with a plurality of apertures therein, or wherein the peripheral portion comprises a mesh or screen.

6.-7. (canceled)

8. The apparatus of claim 1, wherein the peripheral portion is adapted to allow fluid flow therethrough.

9. The apparatus of claim 1, wherein the central portion includes a separator forming a plurality of spaces in an interior of the central portion for accommodating conduits within the housing.

10. (canceled)

11. The apparatus of claim 9, wherein at least one of the conduits is adapted for transmitting fluid flow to or from the interior of the central portion, wherein the separator is adapted to position the at least one of the conduits adapted for transmitting fluid flow away from an inner wall of the central portion to prevent contact between the conduit and a falling film of liquid running down the inner wall.

12. (canceled)

13. The apparatus of claim 1, wherein the central portion of the support forms a container for containing an agitator.

14.-27. (canceled)

27. The apparatus of claim 1, further including an upper frame for positioning above the fixed bed, wherein the upper frame is of a sufficient height to form at least one pocket for allowing fluid to accumulate therein upon exiting an upper end of the fixed bed, and a sensor for sensing a characteristic of the fluid in the at least one pocket.

28.-31. (canceled)

32. The apparatus of claim 1, further including a lid removably connected to the housing, wherein the lid includes a depending portion for engaging the fixed bed or the fixed bed support.

33.-38. (canceled)

39. An apparatus for culturing cells, comprising:
a housing and lid together defining a container having an interior compartment; and
an assembly for positioning within the interior compartment, the assembly including a fixed bed adapted for culturing cells, wherein the assembly is adapted to interlock with the container to retain the position of the fixed bed within the interior compartment.

40. The apparatus of claim 39, wherein the assembly comprises an upper portion adapted to interlock with the lid.

41. The apparatus of claim 40, wherein the upper portion comprises an upper frame for positioning above the fixed bed, wherein the upper frame is of a sufficient height to form at least one pocket for allowing fluid to accumulate therein upon exiting an upper end of the fixed bed, and a sensor for sensing a characteristic of the fluid in the at least one pocket.

42. (canceled)

43. The apparatus of claim 41, wherein the upper frame includes a recess for engaging a projection extending from a lid of the bioreactor.

44. The apparatus of claim 41, wherein the upper frame forms a plurality of pockets, the plurality of pockets each having a different volume.

45. The apparatus of claim 41, wherein the fixed bed comprises a plurality of fixed bed portions, each associated with one of a plurality of supports adapted to interlock with an adjacent support, wherein the upper frame is adapted to interlock with at least one of the plurality of supports.

47. The apparatus of claim 45, wherein each of the plurality of supports comprises a central portion and a peripheral portion adapted to allow fluid to reach the fixed bed, the peripheral portion having an outer diameter corresponding to an inner diameter of the housing.

48. The apparatus of claim 45, further including at least one O-ring between at least two of the plurality of supports or between the lid and at least one of the plurality of supports, wherein, upon attachment of the lid to the housing, the lid is adapted to provide downward pressure on the assembly sufficient to maintain the at least one O-ring in place without glue.

49-50. (canceled)

51. The apparatus of claim 39, wherein the assembly comprises a lower portion for containing an agitator, the lower portion adapted to interlock with at least one support for supporting the fixed bed.

52.-58. (canceled)

59. An apparatus for culturing cells, comprising:
a single piece housing;
at least one fixed bed for culturing cells; and
a plurality of fixed bed supports adapted to interlock for positioning within the single piece housing.

60. The apparatus of claim 59, wherein each of the plurality of fixed bed supports comprises an annular portion and a support frame extending radially outward from the annular portion, wherein support frame comprises an outer diameter that corresponds to an inner diameter of the single piece housing, the support frame comprises a generally planar extension, and the support frame is adapted to allow fluid to flow therethrough.

61.-79. (canceled)
